(19) United States
(12) Patent Application Publication
(10) Pub. No.: US 2017/0087180 A1
(43) Pub. Date: Mar. 30, 2017

(54) METHODS AND COMPOSITIONS FOR TREATING NITRIC OXIDE DEFICIENCY DISORDERS AND RELATED CONDITIONS

(71) Applicants: Sulfagenix, Inc., Chesterland, OH (US); The Board of Supervisors of Louisiana State University and Agricultural and Mechanical College, Baton Rouge, LA (US)

(72) Inventors: Anthony GIORDANO, Chesterland, OH (US); David J. LEFER, New Orleans, LA (US)

(21) Appl. No.: 15/315,651

(22) PCT Filed: Jun. 2, 2015
(86) PCT No.: PCT/US15/33712

§ 371 Date: Dec. 1, 2016

Related U.S. Application Data
(60) Provisional application No. 62/007,100, filed on Jun. 3, 2014.

(51) Int. Cl. A61K 33/04; A61K 9/00; A61K 45/06; A61K 9/48

(52) U.S. Cl. CPC A61K 33/04; A61K 9/48; A61K 9/0014; A61K 45/06

(57) ABSTRACT

The present invention relates to sulfur compositions which are useful, for example, for treating a NO deficiency disorder, supplementing deficits in circulating nitrite, and/or improving bodily endurance or exercise performance.

### BACKGROUND OF THE INVENTION

The present invention relates to the use of sulfur compositions in treating nitric oxide deficiency disorders and related conditions.

Nitric oxide (NO) is a gaseous signaling molecule with fundamental actions in biology. In higher vertebrates it has key roles in maintaining homeostasis and in smooth muscle (especially vascular smooth muscle), neurons and the gastrointestinal tract. NO is involved in regulating aspects from waking, digestion, sexual function, perception of pain and pleasure, memory recall and sleeping. The way NO functions in the body influences how humans degenerate with age. NO also plays a key role in cardiovascular disease, stroke, diabetes, and cancer. Thus, the ability to control NO signaling and to use NO effectively in therapy presents a major bearing on the future quality and duration of human life.

Accordingly, there is a continuing need for both therapeutic and nutritional strategies for maintaining a balanced source of NO for the treatment of conditions associated with NO deficiencies and to promote general health.

### SUMMARY OF THE INVENTION

In one aspect, the present invention features a method of treating a nitric oxide (NO) deficiency disorder in a subject in need thereof (e.g., in a subject in which the level of circulating nitrite is less than about 0.3 μM in the fasted state) by administering or providing an effective amount of a sulfur composition to the subject. NO deficiency disorders that can be treated include endothelial dysfunction, argininosuccinic aciduria, Huntington's disease, sickle cell disease, hyperhomocystinemia, acute chest syndrome, muscular dystrophy, dyslipidemia, and senescence.

In a second aspect, the invention features a method of supplementing deficits in circulating nitrite in a subject in need thereof (e.g., a subject in which the level of circulating nitrite is less than about 0.3 μM in the fasted state). This method includes the step of administering or providing an effective amount of a sulfur composition to the subject.

In a third aspect, the invention features a method of improving bodily endurance or exercise performance in a subject in need thereof, the method including administering or providing an effective amount of a sulfur composition to the subject. Desirably, the composition is administered to the subject prior to or during exercise. In one embodiment, the composition is administered to the subject within 0-180 minutes prior to exercise. In other embodiments, the composition is administered to the subject during exercise. In yet another embodiment, the level of circulating nitrite in the subject is less than about 0.3 μM in the fasted state.

In a fourth aspect, the invention features a method of treating a cardiovascular disease in a subject identified as having a low level of circulating nitrite, the method including: (a) determining the nitrite level in the subject, wherein a level of circulating nitrite in the subject less than about 0.3 μM in the fasted state identifies the subject as having a low level of circulating nitrite, and (b) administering or providing an effective amount of a sulfur composition to the subject.

In some embodiments of the above aspect, the cardiovascular disease is selected from the group consisting of: arteriosclerosis, coronary heart disease, ischemia, endothelium dysfunction, in particular those dysfunctions affecting blood vessel elasticity, restenosis, thrombosis, angina, high blood pressure, cardiomyopathy, hypertensive heart disease, heart failure, cor pulmonale, cardiac dysrhythmias, endocarditis, inflammatory cardiomegaly, myocarditis, myocardial infarction, valvular heart disease, stroke and cerebrovascular disease, aortic valve stenosis, congestive heart failure, and peripheral arterial disease.

In a fifth aspect, the invention features a method of treating a condition associated with oxidative stress in a subject identified as having a low level of circulating nitrite by (a) determining the nitrite level in the subject, wherein a level of circulating nitrite in the subject less than about 0.3 μM in the fasted state identifies the subject as having a low level of circulating nitrite, and (b) administering or providing an effective amount of a sulfur composition and one or more pharmaceutically acceptable excipient.

In certain embodiments of the above aspect, the condition associated with oxidative stress is selected from the group consisting of: schizophrenia, bipolar disorder, fragile X syndrome, sickle cell disease, chronic fatigue syndrome, osteoarthritis cataract, macular degeneration, toxic hepatitis, viral hepatitis, cirrhosis, chronic hepatitis, oxidative stress from dialysis, renal toxicity, kidney failure, ulcerative colitis, bacterial infection, viral infections, such as HIV and AIDS, herpes, ear infection, upper respiratory tract diseases, balding and hair loss, male infertility, over-training syndrome related to athletic performance, athlete's foot, psoriases, eczema, scleroderma, atopic dermatitis, polymyositis, rosacea, dermatitis herpetiformis, other neurodegenerative diseases, other inflammatory disease, and a cancer. In one embodiment of any of the above aspects, the level of circulating nitrite is between about 0.1 μM to 0.3 about μM in the fasted state. In a second embodiment, administration of the composition results in an increase in plasma nitrite concentration within about two hours after administration of the composition. In a third embodiment, the plasma nitrite concentration is maintained between about 0.5 μM to about 1 μM for 2-12 hours. In a fourth embodiment, the administration of the composition to the subject provides for gradual release of the sulfur composition over 4-14 hours.

In any of the above aspects, the sulfur composition can include at least one of hydrogen sulfide, sodium hydrogen sulfide, sodium sulfide, potassium sulfide, calcium sulfide, sodium hydrosulfide dihydrate, sodium sulfide nonahydrate, ammonium sulfide, sodium thiosulfate, potassium thiosulfate, sodium thiosulfate pentahydrate, magnesium thiosulfate, silver thiosulfate, ammonium thiosulfate, calcium dithionate, barium dithionate dihydrate, sodium trithionate, sodium tetrathionate, zinc sulfoxylate, zinc dithionite, sodium dithionite, sodium dithionite dehydrate, or elemental sulfur and one or more highly polar components. In certain aspects of the above features, the composition is administered between one to four times a day.

For any of the methods of the invention, the sulfur composition is desirably administered or provided for a duration sufficient to treat the subject. In one embodiment, the composition is administered for at least two to ten days (e.g., at least two, three, four, five, six, seven, eight, nine, or ten or more days).

In certain embodiments, the composition is formulated for enteral, topical, or parenteral administration. In a particular embodiment, the composition is formulated for enteral administration and the sulfur composition is present in an amount of about 200 mg to about 800 mg. In particular aspects of the embodiment, the composition is a capsule. In certain embodiments, the composition is formulated for topical administration and the composition includes from about 1% to about 20% sulfur composition. In particular aspects of the embodiment, the composition is a cream.

The methods of the present invention optionally further include administering a second agent. In particular embodiments, the second agent is a cardiovascular disease drug, an anti-inflammatory drug, an anti-neurodegenerative drug, or an anti-cancer/anti-proliferative drug. In other embodiments, the second agent is a dietary supplement selected from the group consisting of: a vitamin, a micronutrient, coenzyme 010, glucosamine, chondroitin sulfate, vinpocetine, pramiracetam, diallyl sulfide, a concentrate, an amino acid, an herb or botanical, and a mineral.

The methods of the present invention optionally further include the step of monitoring the plasma nitrite concentration in a sample from the subject, wherein a plasma nitrite concentration greater than about 0.35 μM indicates an improvement in the NO deficiency disorder, deficit in circulating nitrite, bodily endurance or exercise performance, cardiovascular disease, or condition associated with oxidative stress. In one embodiment, if the plasma nitrite concentration in a sample from the subject is below a threshold level, a second dose of the sulfur composition is administered to the subject. In another embodiment, if the plasma nitrite concentration in a sample from the subject is above threshold level, the dose of the sulfur composition is adjusted such that a lower dose of the sulfur composition is administered to the subject or the subject is not administered the sulfur composition.

### DEFINITIONS

“Sulfur composition,” as defined in the current invention, encompasses 1) sulfide compounds, 2) thiosulfate compounds, 3) thionate compounds, 4) thionite compounds, and 5) chemical species capable of providing to a subject in need thereof a therapeutically effective or prophylactically effective amount of a sulfide compound, thiosulfate compound, thionite compound, or thionate compound upon administration to a subject. Non-limiting examples of such chemical species include elemental alpha sulfur, which is known to be biotransformed into a sulfide compound (hydrogen sulfide), and/or conjugates which are known to be chemically and/or enzymatically transformed to sulfide compounds, thiosulfate compounds, thionite compounds, and/or thionate compounds. The sulfur compositions of the invention are characterized as “active” sulfur compositions for the ability of the composition to be biotransformed into hydrogen sulfide upon administration.

As used herein, “sulfide compounds” are compounds formally containing the divalent Sn moiety (S=sulfur; n=1, 2, 3 . . . ) chemically bonded to hydrogen and/or a metal (or metals) and/or a polyatomic cation (or cations) such as hydrogen sulfide, hydrogen disulfide, hydrogen tetrasulfide, sodium hydrosulfide, sodium hydrosulfide dihydrate, sodium sulfide, sodium sulfide nonahydrate, potassium sulfide, calcium sulfide, iron(II) sulfide, silicon (IV) sulfide, zinc sulfide, bismuth (III) sulfide, sodium disulfide, magnesium disulfide, iron (II) disulfide, sodium tetrasulfide, barium tetrasulfide, potassium pentasulfide, cesium hexasulfide, potassium iron (III) sulfide, ammonium sulfide, ammonium disulfide, and ammonium tetrasulfide. Sulfide compounds can also include isotopes of sulfur. Isotopes of sulfur, including but not limited to ³²S, ³³S, ³⁴S, and ³⁶S.

As used herein, “thiosulfate compounds” are compounds formally containing the divalent thiosulfate moiety (S₂O₃) chemically bonded to hydrogen and/or a metal (or metals) and/or a polyatomic cation (or cations), such as sodium thiosulfate (Na₂S₂O₃), potassium thiosulfate (K₂S₂O₃), sodium thiosulfate pentahydrate (Na₂S₂O₃.5H₂O), magnesium thiosulfate (MgS₂O₃), silver thiosulfate (Ag₂S₂O₃), ammonium thiosulfate ((NH₄)₂S₂O₃), and the like. Thiosulfate compounds can also include isotopes of sulfur. Isotopes of sulfur, including but not limited to ³²S, ³³S, ³⁴S, and ³⁶S.

As used herein, “thionate compounds” are compounds formally containing the divalent SₙO₆ (wherein n>1) moiety chemically bonded to hydrogen and/or a metal (or metals) and/or a polyatomic cation (or cations), such as calcium dithionate (CaS₂O₆), barium dithionate dihydrate (BaS₂O₆.2H₂O), sodium trithionate, sodium tetrathionate and the like. Thionate compounds can also include isotopes of sulfur. Isotopes of sulfur, including but not limited to ³²S, ³³S, ³⁴S, and ³⁶S.

As used herein, “thionite compounds” are compounds formally containing the divalent SₙO₂ₙ (wherein n=1 or 2) moiety chemically bonded to a hydrogen and/or a metal (or metals) and/or a polyatomic cation (or cations) such as zinc sulfoxylate, zinc dithionite, sodium dithionite, sodium dithionite dihydrate, and the like. Thionite compounds can also include isotopes of sulfur. Isotopes of sulfur, including but not limited to ³²S, ³³S, ³⁴S, and ³⁶S.

By “elemental sulfur” is meant a chemical element with the symbol S and atomic number 16. Elemental sulfur can exist in over 30 solid allotropes, which are described in Steudel et al., Top Curr Chem 230:1-80 (2003). Exemplary elemental sulfur includes but is not limited to: alpha sulfur, beta sulfur, gamma sulfur, delta sulfur, epsilon sulfur, zeta sulfur, eta sulfur, theta sulfur, iota sulfur, kappa sulfur, lambda sulfur, mu sulfur, nu sulfur, xi sulfur, omicron sulfur, pi sulfur, rho sulfur, tau sulfur, phi sulfur, chi sulfur, psi sulfur, and omega sulfur. Elemental alpha sulfur is a form of orthorhombic elemental sulfur having the formula S₈. Elemental alpha sulfur exists as S₈ (cyclooctasulfur molecules), but can also include S₇ (cycloheptasulfur molecules) and S₆ (cyclohexasulfur molecules). Elemental beta sulfur is a monoclinic elemental sulfur having the formula S₈ and consisting mainly of cyclooctasulfur molecules.

By “highly polar component” is meant a compound whose molecules contain at least one ionic bond or one highly polar covalent bond. Highly polar components include, e.g., sodium polythionates, potassium polythionates, ammonium polythionates, calcium polythionates, sodium thiosulfate, potassium thiosulfate, ammonium thiosulfate, calcium thiosulfate, sodium sulfate, potassium sulfate, ammonium sulfate, sodium bisulfite, potassium bisulfite, ammonium bisulfite, calcium bisulfite, sodium chloride, potassium chloride, ammonium chloride, calcium chloride, sodium acetate, sodium palmitate, potassium palmitate, and/or ammonium laurate. Highly polar components also include molecules containing highly polar O—H covalent bonds, e.g., alcohols, polyols, polythionic acids, carboxylic acids, and/or sorbitan monostearate. Highly polar components further include compounds whose molecules contain highly polar N—H covalent bonds, for example, primary amines, amino acids, primary amides, peptides and proteins.

By “polythionate” is meant an anion or group of the formula ⁻O₃S—Sₙ—SO₃⁻ (e.g., where n is an integer from 1 to 60, preferably from 1-20, and more preferably 1, 2, 3, 4, 5, or 6).

By “zerovalent sulfur” is meant a sulfur atom with an oxidation state of zero, as calculated according to an agreed-upon set of rules well known to a person skilled in the art (e.g., each cyclooctasulfur molecule (S₈) contains eight zerovalent sulfur atoms, each thiosulfate ion (S₂O₃⁻²) contains one zerovalent sulfur atom, and each polythionate ion contains “n” zerovalent sulfur atoms. Zerovalent sulfur can be found in sulfane sulfur compounds.

By “zerovalent sulfur content” is meant the amount of zerovalent sulfur present in a composition containing elemental sulfur.

By “sulfane sulfur” is meant a labile, highly reactive sulfur atom at a reduced oxidation state with a valence of 0 or -1, covalently bound to another sulfur atom. Sulfane sulfur compounds can include, e.g., persulfides, polysulfides, polythionates, polysulfanes, thiotaurine, thiosulfate, and/or elemental sulfur. Sulfane sulfur compounds can be formed in the anaerobic cysteine sulfur metabolism with the participation of enzymes such as cystathionase, 3-mercaptopyruvate sulfurtransferase, and/or rhodanese. The last step in enzymatic H₂S-generating pathways generally involves sulfane sulfur-containing species. Compounds containing sulfane sulfur can participate in cell regulation processes through activation or inactivation of enzymes such as, e.g., oxidoreductase containing an iron or molybdenum atom, e.g., xanthine oxidase, aldehyde oxidase, and malate dehydrogenase).

By “enteral” is meant administration that involves any part of the gastrointestinal tract. Enteral administration can include: by mouth in the form of tablets, capsules, or drops, by gastric feeding tube, duodenal feeding tube, or rectally.

By “threshold level” is meant a level above which indicates an improvement in the disorder or condition (e.g., a NO deficiency disorder, cardiovascular disease or condition associated with oxidative stress). A level above threshold can be for example, a plasma nitrite concentration that is greater than about 0.35 μM (e.g., greater than 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, or 1 μM). A level below threshold can be for example, a plasma nitrite concentration that is less than about 0.35 (e.g., less than 0.3 μM, 0.25 μM, 0.2 μM, 0.15 μM, 0.1 μM, 0.05 μM, or 0.01 μM).

By “topical” is meant administration that is local or systemic, particularly epicutaneous, inhalational, eye drops, and/or ear drops.

By “parenteral” is meant administering the composition of the invention by means other than oral intake, particularly by injection of a form of liquid into the body. Parenteral administration can include:

intravenous, intra-arterial, intraosseous infusion, intra-muscular, intracerebral, intracerebroventricular, and subcutaneous administration.

By “cardiovascular disease drug” is meant a class of agents or substances that are used to treat diseases that affect the cardiovascular system, particularly cardiac disease, vascular disease of the brain and kidney, and peripheral arterial disease.

By “anti-inflammatory drug” is meant an agent or substance that act by reducing inflammation.

By “anticancer/anti-proliferative drug” is meant an agent, substance, and/or mixture of substances that reduces, prevents, and/or interferes with the uncontrolled growth of cells, its initiation, promotion, progression, and/or spread to other organs.

By “anti-neurodegenerative drug” is meant an agent, substance, and/or mixture of substances that restores and/or improves neuron function by acting directly on neurons or indirectly on pathways associated with neuronal function (e.g., axonal transport pathways, protein misfolding pathways, protein degradation pathways, and programmed cell death pathways).

By “dietary supplement” is meant an agent, substance, and/or mixture of substances that is a food supplement or nutritional supplement intended to supplement the diet and provide nutrients, such as vitamins, minerals, fiber, fatty acids, or amino acids that may be missing or may not be consumed in sufficient quantities in a person's diet.

The terms “gradual release,” “sustained release,” or “extended release,” interchangeably refer to a drug formulation that provides for gradual release of a drug over an extended period of time, e.g., 4-14 hours or more (e.g., 4-6 hours, 4-8 hours, 4-10 hours, 4-12 hours, 4-14 hours, 4-18 hours, 4-20 hours, or 4-24 hours) compared to an immediate release formulation of the same drug.

By “inorganic” is meant a compound that is not an organic compound.

By “hydrogen sulfide” is meant a compound having the formula H₂S that is produced in small amounts by many cells of the mammalian body and has a number of biological signaling functions (e.g., a relaxant of smooth muscle, a vasodilator, increases response of NMDA receptor, facilitates long term potentiation, and involvement in memory formation).

By “nitric oxide-deficient subject” is meant a subject with a low level of circulating nitrite.

By “low level of circulating nitrite” is meant a level that is below normal or average (e.g., at least two fold below normal, e.g., 2-fold, 3-fold, 4-fold, or 5-fold, or 2%, 4%, 6%, 8%, 10%, 15%, 20%, 40%, 50%, 80% below normal. In some instances a low level of circulating nitrite is one that is less than 0.3 μM (e.g., less than 0.29 μM, 0.26 μM, 0.24 μM, 0.23 μM, 0.22 μM, 0.2 μM, 0.18 μM, 0.16 μM, 0.14 μM, 0.12 μM, 0.10 μM, 0.008 μM, 0.006 μM, 0.004 μM, or 0.002 μM) or between about 0.1 μM to about 0.3 μM (e.g., 0.1-0.15 μM, 0.1-0.18 μM, 0.1-0.2 μM, 0.2-0.24 μM, 0.2-0.28 μM, 0.2-0.3 μM, 0.25-0.3 μM).

By “control,” “reference,” or “baseline” is meant any sample, standard, or level that is used for comparison purposes. For diagnostic or therapeutic monitoring purposes, a control sample may be a prior sample taken from the same subject (e.g., a sample from a prior time point or prior to the onset of symptoms). Non-limiting examples of control samples include: a sample from a subject or group of subjects not having a NO deficiency disorder or condition associated with oxidative stress; a subject or group of subjects that have no history of a NO deficiency disorder or condition associated with oxidative stress; a sample of a purified reference plasma nitrite at a known normal concentration (i.e., not indicative of a NO deficiency or oxidative stress condition). Additional examples of control samples can be prepared from a subject or group of subjects prior to developing or diagnosis with a NO deficiency or oxidative stress. By “control standard or level” or “normal reference standard or level” is meant a value or number derived from a control sample. For example, a control standard or level can be a value or number derived from a normal subject or group of subjects that is matched to the sample subject, for example, by at least one of the following criteria: age, sex, weight, prior diagnosis of a NO deficiency or condition associated with oxidative stress or any conditions described herein, and a family history of a NO deficiency, condition associated with oxidative stress, or any condition described herein. A “positive control” or “positive reference” sample, standard or value is a sample or value or number derived from a subject or group of subjects that is known to have a NO deficiency or condition associated with oxidative stress.

By “sample” is meant a bodily fluid (e.g., blood, serum, plasma, or saliva) obtained from a subject.

By “increase in plasma nitrite concentration” is meant an increase in the level of nitrite in the plasma (e.g., by at least about 5%, 10%, 20%, 30%, 40%, 50%, 60%, 70%, 80%, 90%, or 100%) as compared to a control reference sample or baseline taken from the same subject. Levels of plasma nitrite can be determined using any useful methods known in the art and those described herein. In some instances, an increase in plasma nitrite concentration can be an increase of greater than about 0.35 μM, desirably greater than about 0.4 μM (e.g., 0.45 μM, 0.5 μM, 0.55 μM, 0.6 μM, 0.65 μM, 0.7 μM, 0.75 μM, 0.8 μM, 0.85 μM, 0.9 μM, 0.95 μM, or 1 μM), or until the plasma nitrite level is maintained between about 0.5 μM to about 1 μM.

By “treating” is meant subjecting a patient to a management regimen for the purpose of combating a disease or disorder and obtaining beneficial or desired results, such as clinical results. Beneficial or desired results can include, but are not limited to, improvement in quality of life, alleviation or amelioration of one or more symptoms or conditions; diminishment of extent of disease, disorder, or condition; stabilization (i.e., not worsening) of a state of disease, disorder, or condition; prevention of spread of disease, disorder, or condition; delay or slowing the progress of the disease, disorder, or condition; amelioration or palliation of the disease, disorder, or condition; and remission (whether partial or total), whether detectable or undetectable.

By “subject” is meant a mammal (e.g., a human or a non-human).

By “effective amount” of an agent is meant the amount of the agent sufficient to effect beneficial or desired result (e.g., treatment of a NO deficiency disorder, cardiovascular diseases, pathological conditions associated with oxidative stress, and an imbalance in redox homeostasis, supplementing deficit in circulating nitrite, and improving bodily endurance and/or exercise performance), and, as such, an amount of the composition sufficient to achieve an increase in in vivo hydrogen sulfide and/or sulfane sulfur levels, as compared to the level of hydrogen sulfide and/or sulfane sulfur without administration of the composition.

By “composition” is meant a system comprising sulfur compounds and/or sulfur elements described herein, optionally formulated with an acceptable excipient, and manufactured or sold with the approval of a governmental regulatory agency as part of a therapeutic regimen for the treatment of disease in a mammal or to promote and maintain general health. Pharmaceutical compositions can be formulated, for example, for oral administration in unit dosage form (e.g., a tablet, capsule, caplet, gel cap, or syrup); for topical administration (e.g., as a cream, gel, lotion, or ointment); for intravenous administration (e.g., as a sterile solution or colloidal dispersion free of particulate emboli and in a solvent system suitable for intravenous use); or in any other formulation described herein.

By “acceptable excipient” is meant any ingredient other than the sulfur compounds and/or sulfur elements described herein (for example, a vehicle capable of suspending or dissolving the sulfur compounds and/or sulfur elements, e.g., petroleum jelly and polyethylene glycol) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, colloid stabilizers, sweeteners, and water. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, ethylcellulose, gelatin, hydroxypropyl cellulose, hydroxypropyl methylcellulose, lactose, magnesium stearate, maltitol, mannitol, methylcellulose, methyl paraben, microcrystalline cellulose, polyethylene glycol, polyvinyl pyrrolidone, povidone, pregelatinized starch, propyl paraben, shellac, silicon dioxide, sodium carboxymethyl cellulose, sodium starch glycolate, sorbitol, starch (corn), stearic acid, sucrose, talc, titanium dioxide, and xylitol. Excipients may also include diluents (e.g., saline and aqueous buffer solutions), aqueous carriers, and nonaqueous carriers, for example, water, ethanol, polyols (such as glycerol, propylene glycol, polyethylene glycol, and the like), and suitable mixtures thereof, vegetable oils, such as olive oil, and injectable organic esters, such as ethyl oleate.

As used herein, the term “about” means±10% of the recited value.

### BRIEF DESCRIPTION OF THE FIGURES

FIG. 1 is a graph showing the plasma hydrogen sulfide levels taken at 0, 0.5, 1, 2, 4, 6, 12, and 24 hours after trial medical food on Day 14 (Visit 3) for 8 healthy subjects.

FIG. 2 is a graph showing the plasma hydrogen sulfide level taken at 0, 0.5, 1, 2, 4, 6, 12, and 24 hours after trial medical food on Day 14 (Visit 3) for a healthy subject.

FIG. 3 is a graph showing the plasma sulfane sulfur level taken at 0, 0.5, 1, 2, 4, 6, 12, and 24 hours after trial medical food on Day 14 (Visit 3) for a healthy subject.

FIG. 4 is a graph showing the plasma nitrite level taken at 0, 0.5, 1, 2, 4, 6, 12, and 24 hours after trial medical food on Day 14 (Visit 3) for a healthy subject.

FIG. 5 is a graph showing the plasma nitrite level taken at 0, 0.5, 1, 2, 4, 6, 12, and 24 hours after an 800 mg dose of trial medical food on Day 14 (Visit 3) for a healthy subject.

### DETAILED DESCRIPTION

We have discovered that administering sulfur compositions results in a sustained increase in plasma nitrite levels in subjects whose baseline levels of circulating nitrite are low. The sustained increase in plasma nitrite levels exhibited by administration of the compositions to these subjects is translates to a maintained increase in NO bioavailability and signaling. Given the observation that exogenous administration of hydrogen sulfide results in an increase and sustained effect in plasma nitrite concentrations thus increasing NO bioavailability, the compositions of the invention may be of benefit for treating conditions or disorders associated with NO deficiency, to supplement a deficit in circulating nitrite in a subject, and to improve bodily endurance and/or exercise performance.

### Methods of Treatment

### NO Deficiency Disorders

The compositions of the invention are useful in treating disorders resulting from NO deficiency or disorders that cause NO deficiency. Reasons for NO deficiency include but are not limited to: 1) NOS dysfunction, resulting in the inability to produce NO from L-arginine in the blood vessels; 2) poor diet with insufficient nitrates and/or excess sugar intake; 3) oral dysbiosis or the inability of oral bacteria to convert dietary sources of nitrate into NO; 4) genetic disorder or weakness that affect NO production (e.g., endothelial dysfunction, argininosuccinic aciduria, Huntington's disease, sickle cell disease, hyperhomocystinemia, acute chest syndrome, muscular dystrophy, dyslipidemia, hypertensive disorders of pregnancy (e.g., pre-eclampsia), or senescence (e.g., Alzheimer's disease)); and 5) sedentary lifestyle.

The compositions of the invention are useful in improving learning and memory related to aging and protecting the skin from sun damage. NO deficiency plays a definite role in aging. Aging can cause >50% loss in endothelial function. Further, a loss of 75% of endothelium derived nitric oxide is seen in 70-80 year old subjects compared to a younger population of subjects. Abnormal vasodilation in certain arteries also occurs with aging. Collectively, these findings illustrate that endothelial function declines progressively with age, as a consequence of declining nitric oxide levels in healthy subjects as well as subjects with existing diseases or disorders. Reduced availability of nitric oxide may increase risk of cardiovascular disease, sexual dysfunction and Alzheimers Disease. Aging impairs the mechanism through which NO in the brain induces sleep. Reduced NO production and impaired endothelia function is observed in obstructive sleep apnea (OSA).

The compositions of the invention are also useful in relieving the symptoms of NO deficiency. Many symptoms of nitric oxide insufficiency occur with age: loss of energy, loss of memory, decline in sexual health and performance, and aches and pains that over time can manifest as specific disease.

### Cardiovascular Diseases

The compositions of the invention are also useful in treating cardiovascular diseases. As used herein cardiovascular diseases included, but are not limited to, arteriosclerosis, coronary heart disease, ischemia, endothelium dysfunction, in particular those dysfunctions affecting blood vessel elasticity, restenosis, thrombosis, angina, high blood pressure, cardiomyopathy, hypertensive heart disease, heart failure, cor pulmonale, cardiac dysrhythmias, endocarditis, inflammatory cardiomegaly, myocarditis, myocardial infarction, valvular heart disease, stroke and cerebrovascular disease, aortic valve stenosis, congestive heart failure, and peripheral arterial disease. In one aspect, the invention includes methods of administering the highly bioavailable zerovalent-sulfur-rich compositions for chronic treatment. In another aspect, the invention also includes methods of administering the highly bioavailable zerovalent-sulfur-rich compositions for acute treatment.

In some embodiments, the compositions of the invention will restore and/or improve cardiovascular parameters to normal ranges in a subject diagnosed with or at risk of a cardiovascular disease. Normal ranges of cardiovascular parameters include but are not limited to, an end-diastolic volume (EDV) from about 65-240 mL, an end-systolic volume (ESV) from about 16-143 mL, a stroke volume from about 55-100 mL, an ejection fraction from about 55-70%, a heart rate from about 60-100 bpm, and/or cardiac output of about 4.0-8.0 L/min.

### Inflammatory Diseases

The compositions of the invention may also be used to treat inflammatory diseases. Examples of inflammatory diseases include, but are not limited to acne vulgaris, asthma, autoimmune diseases (e.g., acute disseminated encephalomyelitis (ADEM), Addison's disease, agammaglbulinemia, alopecia areata, amyotrophic lateral sclerosis, ankylosing spondylitis, antiphospholipid syndrome, antisynthetase syndrome, atopic allergy, atopic dermatitis, autoimmune aplastic anemia, autoimmune cardiomyopathy, autoimmune enteropathy, autoimmunehemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo concentric sclerosis, Behcet's disease, Berger's disease, Bickerstaff's encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's syndrome, cutaneous leukocytoclastic vasculitis, Dego's disease, Dercum's disease, dermatitis herpetiformis, dermatomyositis, diabetes mellitus type 1, diffuse cutaneous systemic sclerosis, Dressler's syndrome, drug-induced lupus, discoid lupus erythematosus, eczema, endometriosis, enthesitis-related arthritis, eosinophilic fasciitis, eosinophilic gastroenteritis, epidermolysis bullosa acquisita, erythema nodosum, erythroblastosis fetalis, essential mixed cryoglobulinemia, Evan's syndrome, fibrodysplasia ossificans progressive, fibrosing alveolitis, gastritis, gastrointestinal pemphigoid, giant cell arteritis, glomerulonephritis, Goodpasture's syndrome, Grave's disease, Guillain-Barre syndrome, Hashimoto's encephalopathy, Hashimoto's thyroiditis, Henoch-Schonlein purpura, herpes gestationis, hidradenitis suppurativa, Hughes-Stovin syndrome, hypogammaglobulinemia, idiopathic inflammatory demyelinating diseases, idiopathic pulmonary fibrosis, idiopathic thrombocytopenic purpura, IgA nephropathy, inclusion body myositis, chronic inflammatory demyelinating polyneuropathy, interstitial cystitis, juvenile idiopathic arthritis, Kawasaki's disease, Lambert-Eaton myasthenic syndrome, leukocytoclastic vasculitis, lichen planus, lichen sclerosus, linear IgA disease, lupus erythematosus, Majeed syndrome, Meniere's disease, microscopic polyangiitis, mixed connective tissue disease, morphea, Mucha-Habermann disease, myasthenia gravis, myositis, narcolepsy, neuromyelitis optica, neuromyotonia, ocular cicatricial pemphigoid, opsoclonus myoclonus syndrome, Ord's thyroiditis, palindromic rheumatism, PANDAS, paraneoplastic cerebellar degeneration, paroxysmal nocturnal hemoglobinuria, Parry Romberg syndrome, Parsonage-Turner syndrome, pars planitis, pemphigus vulgaris, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriatic arthritis, pyoderma gangrenosum, pure red cell aplasia, Rasmussen's encephalitis, raynaud phenomenon, relapsing polychondritis, Reiter's syndrome, restless leg syndrome, retroperitoneal fibrosis, rheumatic fever, Schnitzler syndrome, scleritis, scleroderma, serum sickness, Sjogren's syndrome, spondyloarthropathy, stiff person syndrome, subacute bacterial endocarditis, Susac's syndrome, Sweet's syndrome, sympathetic ophthalmia, Takayasu's arteritis, temporal arteritis, thrombocytopenia, Tolosa-Hunt syndrome, transverse myelitis, ulcerative colitis, undifferentiated connective tissue disease, undifferentiated spondyloarthropathy, vitiligo, and Wegener's granulomatosis), celiac disease, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, interstitial cystitis, and osteoarthritis.

Other Pathological Conditions Associated with Oxidative Stress and/or an Imbalance in Redox Homeostasis

The compositions of the invention may be useful in treating other conditions associated with oxidative stress including but not limited to autism, schizophrenia, bipolar disorder, fragile X syndrome, sickle cell disease, chronic fatigue syndrome, osteoarthritis cataract, macular degeneration, toxic hepatitis, viral hepatitis, cirrhosis, chronic hepatitis, oxidative stress from dialysis, renal toxicity, kidney failure, ulcerative colitis, bacterial infection, viral infections, such as HIV and AIDS, herpes, ear infection, upper respiratory tract diseases, hypertension, balding and hair loss, over-training syndrome related to athletic performance, eczema, scleroderma, atopic dermatitis, polymyositis, and dermatitis herpetiformis.

### Diabetes

Compositions of the invention may also be useful for treating diabetes and its complications. Diabetes can be any metabolic disease in which a person has high blood sugar, either because the body does not produce enough insulin, or because cells do not respond to the insulin that is produced. Non-limiting examples of diabetes includes, type 1 diabetes mellitus, type 2 diabetes mellitus, gestational diabetes, congenital diabetes, cystic fibrosis-related diabetes, steroid diabetes, latent autoimmune diabetes of adults, and monogenic diabetes. Complications associated with diabetes include but are not limited to hypoglycemia, diabetic ketoacidosis, nonketotic hyperosmolar coma, cardiovascular disease, chronic renal failure, diabetic nephropathy, diabetic neuropathy, diabetes-related foot problems (e.g., diabetic foot ulcers), and diabetic retinopathy.

### Cancers

Other conditions that may be treated using compositions of the invention include cancers. Cancers are generally characterized by unregulated cell growth, formation of malignant tumors, and invasion to nearby parts of the body. Cancers may also spread to more distant parts of the body through the lymphatic system or bloodstream. Cancers may be a result of gene damage due to tobacco use, certain infections, radiation, lack of physical activity, obesity, and/or environmental pollutants. Cancers may also be a result of existing genetic faults within cells to cause diseases due to genetic heredity. Screenings may be used to detect cancers before any noticeable symptoms appear and treatment may be given to those who are at higher risks of developing cancers (e.g., people with a family history of cancers). Examples of screening techniques for cancer include but are not limited to physical examination, blood or urine tests, medical imaging, and/or genetic testing. Non-limiting examples of cancers include: bladder cancer, breast cancer, colon and rectal cancer, endometrial cancer, kidney or renal cell cancer, leukemia, lung cancer, melanoma, Non-Hodgkin lymphoma, pancreatic cancer, prostate cancer, ovarian cancer, stomach cancer, wasting disease, and thyroid cancer.

### Bodily Endurance and Exercise Performance

Compositions of the invention are also useful in increasing NO in the body which can significantly boost stamina during high-intensity exercise. Administration of the composition can allow subjects to exercise up to 20% longer and can produce a 1-2% improvement in race time. The compositions of the invention can also significantly reduce systolic blood pressure and reduce the oxygen cost of exercise. Subjects who may benefit from the compositions of the invention may also include those who work out aggressively doing intense workouts, extreme body building and long cardiovascular exercises. The increase in nitrite levels results in an overall increase in NO. NO functions as a vasodilator to help move oxygen into the muscles where it is most needed, stimulating muscle growth, strengthening the gains from exercise and help to produce toned muscles. Replacing NO lost during these workouts is beneficial as it increases energy levels. Administration of the compositions of the invention can increase NO supplies to speed up the blood supply that carries the nutrients to repair and maintain muscles during exercise.

Because NO functions on a localized basis, it is released by different cells throughout the body, enhancing overall functioning. The longer NO circulates in the body, the greater benefit it provides to the cells, cardiovascular system, lungs, nervous system, and organs and the more optimal their functionality will be. The more efficiently the cells functions, the more a subject will be able to produce peak speed, strength, and endurance as part of an athletic endeavor. Furthermore, the desirable effects of NO are not limited to athletes, an increase in NO can promote a pro-wellness lifestyle for all people, ranging from athletes to the sedentary. The compositions of the invention provide a stable and sustained level of nitrite which translates to a stabilized reservoir of NO resulting in continual pro-wellness affects.

### Therapeutic and Prophylactic Use

The compositions containing an effective amount can be administered for prophylactic or therapeutic treatments. In prophylactic applications, compositions can be administered to a patient with a clinically determined predisposition or increased susceptibility to development of a NO deficiency disorder, cardiovascular diseases, hyperproliferative diseases (e.g., cancer), inflammatory diseases, diabetes, dyslipidemia, and other pathological conditions associated with oxidative stress, an imbalance in redox homeostasis, immune dysfunction, and/or endothelia dysfunction. Compositions of the invention can be administered to the patient (e.g., a human) in an amount sufficient to delay, reduce, or preferably prevent the onset of the clinical disease. In therapeutic applications, compositions are administered to a patient (e.g., a human) already suffering from a NO deficiency disorder, cardiovascular disease, hyperproliferative diseases (e.g., cancer), an inflammatory disease, diabetes, dyslipidemia, and other pathological conditions associated with oxidative stress, an imbalance in redox homeostasis, immune dysfunction, and/or endothelial dysfunction, in an amount sufficient to cure or at least partially arrest the symptoms of the condition and its complications. An amount adequate to accomplish this purpose is defined as a “therapeutically effective dose,” an amount of a compound sufficient to substantially improve some symptom associated with a disease or a medical condition. For example, in the treatment of a NO deficiency disorder, cardiovascular disease, hyperproliferative diseases (e.g., cancer), an inflammatory disease, diabetes, dyslipidemia, and other pathological conditions associated with oxidative stress, an imbalance in redox homeostasis, immune dysfunction, and/or endothelia dysfunction, an agent or composition which decreases, prevents, delays, suppresses, or arrests any symptom of the disease or condition would be therapeutically effective. A therapeutically effective amount of an agent or composition is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual.

The compositions and formulations of the present invention may be used in combination with either conventional methods of treatment or therapy or may be used separately from conventional methods of treatment or therapy. When the compositions and formulations of this invention are administered in combination therapies with other agents, they may be administered sequentially or concurrently to an individual. Alternatively, pharmaceutical compositions according to the present invention include a combination of a sulfur compound and/or elemental sulfur or formulation of the present invention optionally in association with a pharmaceutically acceptable excipient, as described herein, and another therapeutic or prophylactic agent known in the art.

### Subject Selection and Assessment of NO Levels

In some embodiments, the subjects are identified as having low levels of circulating nitrite. In certain aspects, prior to administration of the compositions of the inventions, the levels of circulating nitrite are measured in subjects using standard methods in the art and those described herein. Subjects whose circulating plasma nitrite levels are less than about 0.3 μM (e.g., about 0.29 μM, 0.26 μM, 0.24 μM, 0.23 μM, 0.22 μM, 0.2 μM, 0.18 μM, 0.16 μM, 0.14 μM, 0.12 μM, 0.10 μM, 0.008 μM, 0.006 μM, 0.004 μM, 0.002 μM or less) or between about 0.1 μM to about 0.3 μM (e.g., 0.1-0.15 μM, 0.1-0.18 μM, 0.1-0.2 μM, 0.2-0.24 μM, 0.2-0.28 μM, 0.2-0.3 μM, 0.25-0.3 μM) are identified and pre-selected for treatment with the compositions of the invention. In other aspects, the circulating nitrite level is measured before the start of treatment and at specified intervals during treatment (e.g., 7 days, 10 days, 14 days, 21 days, or 24 days after start of treatment) and at the end of the treatment period. In certain embodiments, the measurement of circulating nitrite is taken during the fasted state (e.g., a diagnostic fast or prolonged fasting (from 8-72 hours depending on age of the subject) conducted under observation for investigation).

In other embodiments, the compositions of the invention are administered to athletes engaging in exercise or athletic performances. The compositions can be administered prior to or during the athletic performance (e.g., a soccer match, a basketball game, a weight lifting competition, or a marathon) or exercise routine. In some embodiments, the composition is administered to the subject within 0-180 minutes prior to exercise (e.g., within 0-20 minutes, 0-30 minutes, 0-60 minutes, 0-90 minutes, 0-120 minutes, or 0-150 minutes). In other embodiments, the composition is administered to the subject during exercise. In some aspects, the athlete has a circulating nitrite level that is less than about 0.3 μM in the fasted state. In this case, the athlete is identified as having a low level of circulating nitrite and will benefit from administration of the compositions of the invention.

NO levels can be indirectly determined by measuring the level of the NO metabolite, nitrite. Nitrite is a well-accepted marker for NO and a stable molecule that is both a source and by product of NO. Exemplary methods for measuring nitrite levels are discussed below and further described in Green et al., Anal Biochem. 126(1):131-8 (1982), Berkels et al., J App Physiol. 90:317-320 (2001), Archer., J. FASEB 7:349-360 (1993), and Sun et al., Sensors. 3:276-284 (2003).

Nitrate and/or nitrite can be quantified directly by UV absorbance measurement, GC-MS, HPLC, ion-selective electrodes and capillary electrophoresis. Total nitrite and nitrate is typically determined by converting nitrate into nitrite followed by measuring total amount of nitrite in the sample. In this widely used practice, nitrite concentration is typically measured by a number of additional well-known methods such as colorimetric Griess assay, fluorescent assays, chemiluminescence assay and electrochemical detection.

In one method, the reconversion of nitrate/nitrite to NO can be measured with an amperometric Clark-type electrode. First, nitrate is enzymatically converted to nitrite with the use of the nitrate reductase. Second, nitrite is reduced to equimolar NO concentrations by an acidic iodide solution. The detection limit of the electrode in an aqueous solution is 2 nmol/l NO. This method provides the ability to assess basal and agonist-stimulated NO releases of different biological models.

In another method, serum nitrite and nitrate levels can be measured with the use of the Greiss reagent as described in Rosselli et al., Hypertension 25:848-853 (1995). Briefly, diluted serum obtained from a subject is incubated with substrate buffer (0.1 mol/L imidazole, 210 μmol/L NADPH, 3.8 μmol/L flavine adenine dinucleotide; pH 7.6) in the presence of nitrate-reductase (Aspergillus niger, 70 IU/L; Boehringer Mannheim) for 45 minutes to convert nitrate to nitrite. Total nitrite is analyzed by reacting the samples with Greiss reagent (58 mmol/L sulfanilamide and 3.8 mmol/L naphthalene-ethylene diamine dihydrochloride in 0.5 mol/L H₃PO₄; Spectroquant, Merck). Reacted samples are treated with 200 μL trichloroacetic acid (1.2 mol/L) and centrifuged for 5 minutes at 8000 g, and the absorbance of the supernatant measured at 525 nm. Amounts of nitrite in serum is estimated from a standard curve of NaNO₂ obtained by enzymatic conversion of NaNO₃ (0 to 32 μmol/L, Merck).

In yet another method, saliva NO test strips can be used to measure NO. Saliva NO Test Strips are hand held strips that rapidly turn from white to deep pink based on the amount of a surrogate marker for Nitric Oxide. The intensity of the color on the Nitric Oxide Test strips is compared to a standard color chart. The NO test strip collects and measures nitrite. On one end of the strip is the saliva absorption pad. The absorption pad is placed on the tongue to collect saliva and the absorption pad is folded against the test pad. The test pad will change colors. If the pad remains white with a hue of light pink, the subject has low NO levels, if the pad shows a deep pinkish-red, NO levels are elevated. This method is generally performed after consumption of a meal rich in nitrate/nitrite.

In particular embodiments, plasma nitrite levels are measured as described in the Examples described below.

### Subject Monitoring

The methods of the present invention may be carried out with methods for monitoring the NO deficiency disorder, condition associated with oxidative stress, cardiovascular disease, or exercise endurance and performance using methods for measuring NO levels known in the art or described herein. The monitoring of the disorder or condition can provide information on the state of the disorder or condition (e.g., worsening or improvement) to facilitate changes in treatment regime. The progression in body endurance and exercise performance can also be monitored using the methods known in the art and described herein. Non-limiting examples of monitoring methods are the measurement and detection of levels of nitrite, nitrate, and/or metabolites of NO other than nitrite/nitrate.

In one example, treatment or supplementation is administered until the blood, plasma, or serum nitrite level is greater than about 0.35 μM, desirably greater than about 0.4 μM (e.g., 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1 μM, or until the plasma nitrite level is maintained between about 0.5 μM to about 1 μM. In any embodiments, the plasma nitrite concentration is measured repeatedly as a method of not only diagnosing a condition or disorder or need for supplementation, but monitoring the treatment and management of the condition or disorder. In one example, the plasma nitrite concentration is measured at a certain time post-treatment with a sulfur composition (e.g., 7 days, 14 days, 21 days, 24 days, 30 days or more after treatment) where if the plasma nitrite concentration is below or above a threshold, the dosage of the sulfur composition is altered (e.g., providing a higher dose, a lower dose, no dose, reducing the frequency of administration, increasing the frequency of administration, reducing the duration of administration, increasing the duration of administration) based on the measured plasma nitrite concentration. In one embodiment, if the plasma nitrite concentration is above a threshold level (e.g., greater than about 0.35 μM, e.g., 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1 μM) a lower dose of sulfur composition is administered (e.g., from 800 mg to 700 mg, 600 mg, 500 mg, 400 mg, 300 mg, 200 mg, 100 mg, or 50 mg), the frequency and/or duration of administration is reduced (e.g., from 4 times a day to 3 times a day, 2 times a day, or one time a day and/or from administration of 7 days to administration of 6 days, 5 days, 4 days, 3 days, 2 days, or one day). In another embodiment, if the plasma nitrite concentration is below a threshold level (e.g., less than about 0.35 μM, e.g., 0.3 μM, 0.25 μM, 0.2 μM, 0.15 μM, 0.1 μM, 0.05 μM, 0.01 μM) a high dose of sulfur composition is administered (e.g., from 50 mg to 100 mg, 200 mg, 300 mg, 400 mg, 500 mg, 600 mg, 700 mg, 800 mg, 1000 mg), the frequencing and or duration of administration is increased (e.g., from one time a day to 2 times a day, 3 times a day, 4 times a day, 5 times a day and/or from administration of 2 days to 3 days, 4, days, 5 days, 6 days, or 7 days or more).

### Sulfur Compositions

### Sulfur Compounds

Sulfur compounds encompass: 1) sulfide compounds, 2) thiosulfate compounds, 3) thionate compounds, 4) thionite compounds, and 5) organic, inorganic or organometallic precursors of sulfide compounds, thiosulfate compounds, thionate compounds, and thionite compounds. The sulfur compounds are considered active as they are readily biotransformed into hydrogen sulfide.

Sulfide compounds are compounds formally containing the divalent Sₙ moiety (S-sulfur; n=1, 2, 3 . . . ) chemically bonded to hydrogen and/or a metal (or metals) and/or a polyatomic cation (or cations) such as hydrogen sulfide, hydrogen disulfide, hydrogen tetrasulfide, sodium hydrosulfide, sodium hydrosulfide dihydrate, sodium sulfide, sodium sulfide nonahydrate, potassium sulfide, calcium sulfide, iron(II) sulfide, silicon (IV) sulfide, zinc sulfide, bismuth (III) sulfide, sodium disulfide, magnesium disulfide, iron (II) disulfide, sodium tetrasulfide, barium tetrasulfide, potassium pentasulfide, cesium hexasulfide, potassium iron (III) sulfide, ammonium sulfide, ammonium disulfide and ammonium tetrasulfide.

Sulfite compounds are compounds formally containing the divalent sulfite moiety (SO₃) chemically bonded to hydrogen, and/or a metal (or metals) and/or a polyatomic cation (or cations), such as sodium sulfite, potassium sulfite, ammonium sulfite, calcium sulfite, and cesium hydrogensulfite.

Thiosulfate compounds are compounds formally containing the divalent thiosulfate moiety (S₂O₃) chemically bonded to hydrogen and/or a metal (or metals) and/or a polyatomic cation (or cations), such as sodium thiosulfate (Na₂S₂O₃), sodium thiosulfate pentahydrate (Na₂S₂O₃.5H₂O), magnesium thiosulfate (MgS₂O₃), silver thiosulfate (Ag₂S₂O₃), ammonium thiosulfate [(NH₄)₂S₂O₃].

Thionate compounds are compounds formally containing the divalent SnO6 (n>1) moiety chemically bonded to hydrogen and/or a metal (or metals) and/or a polyatomic cation (or cations), such as calcium dithionate (CaS₂O₆), barium dithionate dihydrate (BaS₂O₆.2H₂O), sodium trithionate and sodium tetrathionate.

Thionite compounds are compounds formally containing the divalent SₙO₂ₙ (n=1 or 2) moiety chemically bonded to a hydrogen and/or a metal (or metals) and/or a polyatomic cation (or cations) such as zinc sulfoxylate, zinc dithionite, sodium dithionite and sodium dithionite dihydrate.

Organic, inorganic or organometallic precursors of the previously defined compounds are any and all chemical species from which sulfide compounds and/or thiosulfate compounds and/or thionite compounds and/or thionate compounds, can arise through chemical change and/or enzyme action and/or biotransformation in a mammal's body. Therefore, tetraphosphorus decasulfide (P₄S₁₀) and sodium thiosilicate (Na₂SiS₃) are precursors of sulfide compounds.

Other sulfur compounds are described in U.S. Pat. No. 8,361,514, U.S. Pat. No. 8,389,005, German patent DE3419686, International Publication No. WO 84/02527, and U.S. Pat. No. 5,045,316, all of which are hereby incorporated by reference. The sulfur compounds can be used in the compositions individually or in admixture with each other and/or with any of the sulfide compounds, thionite compounds, or thionate compounds described herein. The sulfur compounds can also include isotopes of sulfur (e.g., ³²S, ³³S, ³⁴S, or ³⁶S).

### Elemental Sulfur

In one embodiment, the sulfur compositions including an elemental sulfur and highly polar components is obtained by the procedure as described in U.S. Patent Publication No. 2013-0064904, which is hereby incorporated by reference in its entirety. The sulfur composition can include about 99% elemental sulfur and about 1% highly polar components (e.g., sodium sulfate and traces of sodium polythionates and sodium thiosulfate). In certain embodiments, the elemental sulfur is elemental alpha sulfur or elemental beta sulfur. Elemental alpha sulfur can contain one or more of a cyclooctasulfur molecule, cycloheptasulfur molecules, and/or a cyclohexasulfur molecule. In yet other embodiments, the elemental sulfur can exist in any of the solid allotropes described in Steudel et al., Top Curr Chem 230:1-80 (2003). Elemental sulfur can include any of the following forms listed in Table 1.

TABLE 1
List of Elemental Sulfur

| | | Molecular | Designation used |
|---|---|---|---|
| Name | Synonyms | species | in this review |
| α (alpha) | Rhombic, | Cycloocta-S | Orthorhombic-α |
| | orthorhombic, | | |
| | Muthmann's I | | |
| β (beta) | Monoclinic I, | Cycloocta-S | Monoclinic-β |
| | Muthmann's II, | | |
| | prismatic | | |
| γ (gamma) | Monoclinic II, | Cycloocta-S | Monoclinic-γ |
| | Muthmann's III, | | |
| | nacreus, mother-of- | | |
| | pearl, Gernez | | |
| δ (delta) | Monoclinic III, | Cycloocta-S | Allotropes of S₈ |
| | Muthmann's IV, | | |
| | γ-monoclinic | | |
| ε (epsilon) | Engel, Aten, rhombo- | Cyclohexa-S | Rhombohedral |
| | hedral, monoclinic | | |
| | Engel | | |
| ζ (zeta) | 5th monoclinic, | Cycloocta-S | Allotrope of S₈ |
| | Korinth | | |
| η (eta) | 4th monoclinic, | Cycloocta-S | Allotrope of S₈ |
| | Korinth | | |
| θ (theta) | Tetragonal, Korinth | Cycloocta-S | Allotrope of S₈ |
| ι (iota) | Erämetsä | Cycloocta-S | Allotrope of S₈ |
| κ (kappa) | Erämetsä | Cycloocta-S | Allotrope of S₈ |
| λ (lambda) | | Cycloocta-S | Cycloocta-S₈ |
| μ (mu) | (a) Insoluble | Catenapoly-S | Solid or liquid |
| | (b) Polymeric | | Polymeric-S |
| ν (nu) | μ | Mixture | Solid polymeric |
| ξ (xi) | Triclinic, Korinth | Cycloocta-S | Allotrope of S₈ |
| ο (omicron) | Erämetsä | Cycloocta-S | Allotrope of S₈ |
| π (pi) | (a) Aten, Erämetsä | Ring mixture | Frozen liquid |
| | (b) Catenaocta-S | | |
| ρ (rho) | Aten, Engel | Cyclohexa-S | Cyclohexa-S |
| τ (tau) | Erämetsä | Cycloocta-S | Allotrope of S₈ |
| φ (phi) | Fibrous | Mixture | Fibrous |
| Φ (phi) | Fibrous, plastic | Polycatena-S | Fibrous |
| χ (chi) | Plastic | Mixture | Polymeric |
| ψ (psi) | Fibrous | Mixture | Fibrous |
| ω (omega) | Insoluble, white, Das | Mixture | Polymeric |
| | supersublimation | | |

### Formulations

When employed as pharmaceuticals, the sulfur composition, including e.g., sulfide compounds, thiosulfate compounds, thionate compounds, thionite compounds, or elemental sulfur, can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

The pharmaceutical composition can contain one or more pharmaceutically acceptable excipients. In making a pharmaceutical composition for use in a method of the invention, the sulfur composition pharmaceutically acceptable salt, solvate, or prodrug thereof is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention (e.g., the sulfur composition) can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 6^{th} Edition, Rowe et al., Eds., Pharmaceutical Press (2009). The pharmaceutical compositions can be formulated so as to provide immediate, extended, or delayed release of the active ingredient after administration to the patient by employing procedures known in the art.

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

The pharmaceutical compositions formulated for oral delivery, such as tablets or capsules of the present invention can be coated or otherwise compounded to provide a dosage form affording the advantage of delayed or extended release. The coating may be adapted to release the active drug in a predetermined pattern (e.g., in order to achieve a controlled release formulation) or it may be adapted not to release the active drug until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug). The coating may be applied on the solid dosage form in a similar manner as that described in Encyclopedia of Pharmaceutical Technology, vols. 5 and 6, Eds. Swarbrick and Boyland, 2000.

The composition can be formulated as a suspension, microemulsion, liposome, or other ordered structure suitable to high drug concentration. The carrier can be a solvent or dispersion medium containing, for example, water, ethanol, petroleum jelly (e.g., Vaseline®), polyol (e.g., glycerol, propylene glycol, and liquid polyethylene glycol, and the like), and suitable mixtures thereof, formulated at different percentages (e.g., 5, 10, 15, 20, 25, 30, 35, 40, 45, or 50% by weight in a dispersion medium described herein). The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Colloidal dispersions may be stabilized through addition of agents well known in the art.

The compositions of the invention may be sterilized by conventional sterilization techniques, or may be sterile filtered. The resulting aqueous dispersions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid or semisolid form may be packaged in multiple single dose units, each containing a fixed amount of the composition, such as in a sealed package of tablets or capsules. The composition in solid form can also be packaged in a container for a flexible quantity, such as in a squeezable tube designed for a topically applicable cream or ointment.

Preferred formulations of the invention include but are not limited to: preparation of hard gelatin capsules containing from about 200 to about 800 mg of a sulfur composition, preparation of a suspension of about 5-20% (5.5%, 6%, 6.5%, 7%, 8%, 10%, 15%, 17%, or 19%) of a sulfur composition and petroleum jelly (e.g., Vaseline®) or polyethylene glycol, or a colloidal dispersion of about 5-20% (5.5%, 6%, 6.5%, 7%, 8%, 10%, 15%, 17%, or 19%) of a sulfur composition in water or oil.

The formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

### Dosage

The pharmaceutical compositions of the present invention are formulated into pharmaceutically acceptable dosage forms by conventional methods known to those of skill in the art. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of absorption of the particular agent being employed, the duration of the treatment, other drugs, substances, and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian can start doses of the sulfur composition of the invention employed in the pharmaceutical composition at levels lower than that required in order to achieve the desired therapeutic effect and gradually increase the dosage until the desired effect is achieved. In general, a suitable daily dose of a composition of the invention will be that amount of the sulfur composition which is the lowest dose effective to produce a therapeutic effect. Such an effective dose will generally depend upon the factors described above. Preferably, the effective daily dose of a therapeutic composition may be administered as two, three, four, five, six or more sub-doses administered separately at appropriate intervals throughout the day, optionally, in unit dosage forms.

The compositions can be formulated in a unit dosage form, each dosage containing, e.g., from about 10 mg to about 1000 mg of the active ingredient (e.g., the sulfur composition). For example, the dosages can contain from about 10 mg to about 50 mg, from about 10 mg to about 40 mg, from about 10 mg to about 20 mg, from about 20 mg to about 100 mg, from about 20 mg to about 200 mg, from about 20 mg to about 400 mg, from about 20 mg to about 600 mg, from about 20 mg to about 800 mg, from about 50 mg to about 150 mg, from about 50 mg to about 250 mg, from about 50 mg to about 350 mg, from about 50 mg to about 450 mg; from about 50 mg to about 650 mg, from about 50 mg to about 750 mg, from about 50 mg to about 800 mg, from about 200 mg to about 400 mg, from about 200 mg to about 600 mg, from about 200 mg to about 800 mg; from about 200 mg from to about 1000 mg, from about 400 mg to about 600 mg, from about 400 mg to about 600 mg, from about 400 mg to about 800 mg, from about 400 mg to about 1000 mg; from about 600 mg to about 800 mg, from about 600 mg to about 1000 mg, from about 800 mg to about 1000 mg of the active ingredient. For preparing solid compositions such as tablets, the principal active ingredient (e.g., the sulfur composition) is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above.

Preferred therapeutic dosage levels are between about 800 mg to about 1600 mg (e.g., 800, 850, 900, 1000, 1050, 1100, 1200, 1300, 1400, 1450, 1500, 1550, and 1600 mg) of a sulfur composition-per day administered orally to adults of average weight afflicted with most of the symptoms and conditions described herein, Preferred prophylactic dosage levels are between about 100 mg to about 1200 mg (e.g., 110, 140, 200, 250, 300, 350, 400, 460, 700, 750, 800, 900, 1000, 1100, and 1150 mg). For children afflicted with cancer, the dose may be titrated (e.g., the dose may be escalated gradually until signs of gastrointestinal toxicity appear, such as diarrhea or nausea). In preferred embodiments, the compositions of the invention are extremely safe for oral administration and most patients can tolerate higher dosages as treatment progresses.

The amount of the sulfur composition per dose can vary. For example, a subject can receive from about 200 mg to about 800 mg of the sulfur composition. Generally, the composition is administered in an amount such that the measured plasma nitrite concentration ranges in a nitrite-deficient subject (e.g., one with plasma nitrite levels below 0.3 μM) are from between 0.35 μM to about 1 μM. Exemplary peak plasma nitrite concentrations can range from 0.35 μM to 0.4 μM, 0.35 μM to 0.45 μM, 0.4 μM to 0.45 μM, 0.4 μM to 0.5 μM, 0.5 μM to 0.6 μM, 0.5 μM to 0.7 μM, 0.5 μM to 0.8 μM, 0.5 μM to 0.9 μM, 0.6 μM to 0.8 μM, 0.6 μM to 0.9 μM, 0.7 μM to 0.8 μM, 0.7 μM to 0.9 μM, 0.8 μM to 0.9 μM, 0.8 μM to 1 μM, or 0.9 μM to 1 μM. The peak plasma concentrations may be maintained for 2-24 hours, 2-12 hours, 2-6 hours, or 2-4 hours.

The frequency of treatment may also vary. The subject can be treated one or more times per day (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic or prophylactic effect, optionally in association with the required pharmaceutical carrier. The specifications for the dosage unit forms of the invention are dictated by and directly dependent on (a) the unique characteristics of the sulfur composition and the particular therapeutic or prophylactic effect to be achieved, and (b) the limitations inherent in the art of compounding such a sulfur composition for the treatment of sensitivity in individuals.

Sterile injectable colloidal suspensions can be prepared by incorporating the active compound in the required amount in an appropriate solvent with one or a combination of ingredients enumerated above, as required, optionally followed by sterilization microfiltration. Generally, dispersions are prepared by incorporating the active compound into a sterile vehicle that contains a basic dispersion medium and the required other ingredients from those enumerated above. Dosage regimens are adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic or prophylactic situation. For example, the compositions of the invention may be administered once or twice weekly by subcutaneous injection or once or twice monthly by subcutaneous injection.

In other embodiments, the compositions of the invention are safe for topical administration. Acceptable dosage forms for topical administration can be formulated as creams, lotions, pastes, gels, and/or ointments containing the highly bioavailable zerovalent-sulfur-rich compositions.

Final dosage forms suitable for administration to human subjects may comprise the sulfur composition as pharmacologically-active agent or further comprise other active agents such as alpha-lipoic acid, carnitine, carnitine tartrate, carnitine fumarate, coenzyme 010, selenium, alpha-ketoglutaric acid, potassium alpha-ketoglutarate, diethyl alpha-ketoglutarate, oxaloacetic acid, sodium oxaloacetate, diethyl oxaloacetate, 2-oxo-3-(ethoxycarbonyl)-pentanodioc acid diethyl ester, L-cystine, paracetamol, a sulfa drug, an NSAID, a corticosteroid, taurine, a vitamin, a prebiotic, another anticancer drug, including but not limited to another mitocan (e.g., a drug targeting the mitochondrial electron transport chain), alkylating agents (e.g. procarbazine, dacarbazine, altretamine, cisplatin), methotrexate, purine antagonists (e.g., mercaptopurine, thioguanine, cladribine, pentostatin), pyrimidine antagonists (e.g., fluorouracil, cytarabine, azacitidine), plant alkaloids (e.g., vinblastine, etoposide, topotecan), hormonal agents (e.g., tamoxifen, flutamide), antibiotics (e.g., doxorubicin, daunorubicin, mitomycin, bleomycin), and mitocans (e.g., sodium dichloroacetate and 3-bromopyruvic acid).

### Medical Food

The present invention also relates to compositions including the sulfur composition as medical food for daily intake and for maintaining and promoting general health. Evidence indicates that daily ingestion by an adult of average weight of about 800 mg of the compositions described herein during extended periods is safe and beneficial to health because it brings about a marked reduction in the frequency and severity of digestive and respiratory infections (e.g., of viral and bacterial origin) and allergic episodes. Daily intake of the composition of the invention is also associated with a greatly reduced probability of being afflicted by cancer, AIDS, a neurodegenerative condition, stroke, diabetes and its complications, cardiovascular disease, and confers protection from cardiovascular, cerebrovascular, gastric, and hepatic damage caused by xenobiotics including, drugs such as paracetamol, corticosteroids, NSAIDs and antiretrovirals, toxins and poisons (e.g., cyanide, thallium, methanol). Daily intake of the composition of the invention can also result in faster growth of hair and nails, firmer skin, a prebiotic-like effect, and a sense of general wellness.

In one aspect, the compositions of the invention are used as a paravitamin to provide a supplemental source of cysteine and its derivatives. Cysteine and its derivatives (e.g., glutathione, taurine, taurine conjugates with bile acids, hydrogen sulfide, and sulfate ions) play a role similar to that of vitamins. Like antioxidative vitamins, cysteine and its derivatives play a role in the oxidant/antioxidant balance and indirectly in the regulation of metabolic processes. Cysteine supplementation on top of the normal diet can have various beneficial effects, for example, cysteine supplementation can lead to an increase in muscle function, immune function, plasma albumin concentration and a decrease in TNF-α concentration. Supplementation can also restore the body's reservoirs of cysteine and glutathione levels and maintain and stabilize nitrite levels, which are driving forces behind multiple ageing-related processes.

In another aspect, the paravitamins are medical foods providing a minimum amount of calories and maximum amount of a bioavailable form of sulfur intended for humans not receiving enough sulfur in their diets. Evidence obtained from in vivo experiments showed that in mammals, hydrogen sulfide, sulfane sulfur, glutathione, and nitrite levels are increased in blood and tissues upon administration of the compositions as paravitamins.

In some embodiments, the compositions of the invention are rapidly and efficiently converted into hydrogen sulfide in the body, which in turn is largely transformed into L-cysteine. L-cysteine may be used as a building block in the synthesis of peptides enzymes, and other proteins and small sulfur-containing biomolecules (e.g., keratin which constitutes 14% of hair and nails, e.g., glutathione, a tripeptide needed for regulating and potentiating immune function and for cellular protection from oxidants, electrophiles, e.g., taurine, which is essential for cardiovascular function, development, and function of skeletal muscle, the retina, the central nervous system, it is a major constituent of bile; has many fundamental biological roles such as conjugation of bile acids, antioxidant, osmoregulation, membrane stabilization, and modulation of calcium signaling, e.g., sulfate, which is necessary for the synthesis of cartilage and for detoxification of many drugs including but not limited to corticosteroids and acetaminophen). In another embodiment, the compositions of the invention are transformed and stored as sulfane sulfur. Sulfane sulfur is conveniently used by the body as highly versatile precursor of hydrogen sulfide which readily releases hydrogen sulfide whenever and wherever this species is needed to activate protective genes, block inflammation, and protect cells from free-radical damage. In another embodiment, the increased levels of sulfane sulfur and/or hydrogen sulfide results in an increase in nitrite levels where nitrite can be a substrate for NOS-independent generation of NO in vivo. High natural levels of NO in subjects are beneficial for high immune responses and better-than-average wound healing. Maintaining a normal dose of NO naturally produced by the body also improves general health, good muscle development, and healthy blood flow. Furthermore, administration of the compositions of the invention as medical food enhances the above areas.

In yet another embodiment, the maximum human life span may be increased beyond the previous limit by providing compositions of the invention as paravitamins, glutathione levels will be restored to a normal level in the cells of the immune system thereby normalizing the function of the immune system and restoring health and well-being.

### Combination Therapies

Pharmaceutical compositions of the invention can be administered in combination therapy, i.e., combined with other agents (e.g., an antiplatelet drug, a β blocker, an ACE inhibitor or ARB, a statin, fibrates, biguanides, blood pressure lowering agents, cytokines, cholesterol lowering agents, erectile dysfunction drugs, anti-inflammatory drugs, anti-thrombosis drugs, anticancer drugs, anti-diabetic drugs, and/or dietary supplements) depending on the condition to be treated.

### Prevention Drugs for Cardiovascular Diseases

Compositions of the invention can be administered in combination with one or more drugs that are used as secondary prevention drugs for cardiovascular diseases. Examples of preventative drugs include, but are not limited to, β blockers (e.g., nonselective agents, e.g., alprenolol, carteolol, oxprenolol, sotalol, timolol, e.g., β₁-selective agents, e.g., acebutolol, betaxolol, celiprolol, metoprolol, e.g., β₂-selective agents, e.g., butaxamine, e.g., β₃-selective agents, e.g., SR 59230A), statins (e.g., atorvastatin, cerivastatin, fluvastatin, lovastatin, mevastatin, pravastatin, simvastatin, and rosuvastatin), fibrates (e.g., bezafibrate, ciprofibrate, clofibrate, gemfibrozil, and fenofibrate), biguanides (e.g., metformin, phenformin, buformin, and proguanil), and/or ACE inhibitors (e.g., sulfhydryl-containing agents, e.g., captopril, zofenopril, e.g., dicarboxylate-containing agents, e.g., enalapril, ramipril, quinapril, perindopril, imidapril, e.g., phosphate-containing agents, e.g., fosinopril).

### Drugs for Treatment of Erectile Dysfunction

The compositions of the invention can be administered in combination with one or more drugs for treatment of erectile dysfunction. Examples of drugs for treatment of erectile dysfunction include, but are not limited to: sildenafil, tadalafil, vardenafil, alprostadil, avanafil, and yohimbine.

### Anti-Neurodegenerative Drugs

The compositions of the invention can be administered in combination with one or more anti-neurodegenerative drugs. Examples of anti-neurodegenerative drugs include, but are not limited to, acetylcholinesterase inhibitors (e.g., donepezil, galantamine, and rivastigmine), anti-glutamate agent (e.g., amantadine, GABA-ergic, valproic acid), reserpine, tetrabenazine, typical/atypical neuroleptics, tricyclic antidepressants, SSRIs, carbamazepine, baclofen, tizanidine, and lamotrigine.

### Anti-Inflammatory Drugs

The compositions of the invention can be administered in combination with one or more anti-inflammatory drugs. Examples of anti-inflammatory drugs include, but are not limited to, steroids (e.g., glucocorticoids, e.g., corticosteroids), non-steroidal anti-inflammatory drugs (NSAIDs) (e.g., aspirin, diflunisal, salsalate, ibuprofen, naproxen, fenoprofen, ketoprofen, flurbiprofen, sulindac, etodolac, ketorolac, nabumetone, piroxicam, meloxicam, tenoxicam, mefenamic acid, flufenamic acid, tolfenamic acid, celecoxib, rofecoxib, parecoxib, etoricoxib, firocoxib, nimesulide, and licofelone), immune selective anti-inflammatory derivatives (ImSAIDs) (e.g., phenylalanine-glutamine-glycine (FEG) and its D-isomeric form (feG)), and/or herbs (e.g., Harpagophytum, hyssop, ginger, turmeric, Arnica montana, and willow bark)

### Dietary Supplements

The composition of the invention can be administered in combination with one or more dietary supplements to promote and/or maintain general health. Examples of dietary supplements include, but are not limited to, a vitamin (e.g., Vitamin A, Vitamin B₁, B₂, B₃, B₅, B₆, B₇, B₉, B₁₂, Vitamin C, Vitamin D, Vitamin E, and Vitamin K), a mineral (e.g., potassium, chlorine, sodium, calcium, magnesium, phosphorus, zinc, iron, manganese, copper, iodine, selenium, methylcobolamin, and molybdenum), an herb or botanical (e.g., St. John's-wort, kava, Shilajit, and Chinese herbal medicines), an amino acid (e.g., glycine, serine, methionine, cysteine, aspartic acid, glutamic acid, glutamine, tryptophan, phenylalanine, and N-acetylcysteine), pyridoxal-S-phosphate, 5-methylcetrahydrofolate, nitrate, and a concentrate, constituent, extract, and/or a combination of any of the above

### Anticancer/Anti-Proliferative Drugs

The compositions of the invention can be formulated or administered in combination with one or more anticancer drugs. Examples of anticancer agents include, but are not limited to: chemotherapeutic agents (e.g., arsenic trioxide, cisplatin, carboplatin, chlorambucil, melphalan, nedaplatin, oxaliplatin, triplatin tetranitrate, satraplatin, imatinib, nilotinib, dasatinib, and radicicol), immunomodulatory agents (e.g., methotrexate, leflunomide, cyclophosphamide, cyclosporine A, minocycline, azathioprine, antibiotics (e.g., tacrolimus), methylprednisolone, corticosteroids, steroids, mycophenolate mofetil, rapamycin, mizoribine, deoxyspergualin, brequinar, T cell receptor modulators, and cytokine receptor modulators), antiangiogenic agents (e.g., bevacizumab, suramin, and etrathiomolybdate), mitotic inhibitors (e.g., paclitaxel, vinorelbine, docetaxel, abazitaxel, ixabepilone, larotaxel, ortataxel, tesetaxel, vinblastine, vincristine, vinflunine, and vindesine), nucleoside analogs (e.g., gemcitabine, azacitidine, capecitabine, carmofur, cladribine, clofarabine, cytarabine, decitabine, floxuridine, fludarabine, fluorouracil, mercaptopurine, pentostatin, tegafur, and thioguanine), DNA intercalating agents (e.g., doxorubicin, actinomycin, bleomycin, mitomycin, and plicamycin), topoisomerase inhibitors (e.g., irinotecan, aclarubicin, amrubicin, belotecan, camptothecin, daunorubicin, epirubicin, etoposide, idarubicin, mitoxantrone, pirarubicin, pixantrone, rubitecan, teniposide, topotecan, valrubicin, and zorubicin), folate antimetabolites (e.g., pemetrexed, aminopterin, methotrexate, pralatrexate, and raltitrexed), mitocans (e.g., sodium dichloroacetate and 3-bromopyruvic acid), and other targeting agents (e.g., agents that target particular enzymes or proteins involved in cancer or agents that target particular organs or types of cancers), and combinations thereof.

### EXAMPLES

The compositions of the invention and their uses will now be illustrated by means of the following non-limiting examples. These examples are set forth merely for illustrative purposes and many other variations may be used.

### Methods

### Measurement of Plasma Nitrite (NO₂)

Plasma nitrite concentrations were quantified by an automated ion chromatography system (ENO30 Analyzer, Eicom). Nitrite was separated by a column (NO-PAK with polystyrene polymer, Eicom). The mobile phase, delivered at a pump rate of 0.33 mL/min, was 10% methanol containing 0.15 mol; L NaCl—NH₄Cl and 0.5 g/L of tetrasodium EDTA. The Griess reagent, which was 1.25% HCl containing 5 g/L sulfanilamide with 0.25 g/L N-naphthylethylenediamine, was delivered at a rate of 0.1 mL/min. For a detailed description of methods for measuring plasma nitrite see, for example, King et al., Proc Natl Acad Sci USA 111(8):3182-7 (2014); Bhushan et al., Circ Res 114(8):1281-91 (2014); Calvert et al., Circ. Res. 108(12):1448-58; Bryan et al., Proc Natl Acad Sci USA 104(48):19144-9 (2007); and Kondo et al., Circulation 127(10):1116-27 (2013).

### Example 1: Phase 1 Study Design and Rationale

SG1002 is described in Kondo et al., Circulation 127(10):1116-27 (2013) and in U.S. Patent Application Publication No. 2013-0064904. SG1002 has been used in a number of cases in children aged 18 months and over and in adults (n=13). These patients received between 1,200 mg and 3,600 mg SG1002 daily for conditions including osteosarcoma (n=2), hydrocephalus with or without tumour (n=6), medullobastoma (n=1), squamous cell carcinoma (n=1), acute lymphobastic leukaemia (n=1), heart failure (n=1) or type 2 diabetes (n=1). No information on adverse events was reported.

There was one placebo-controlled randomized study of SG1002,500 mg daily compared to resveratrol 50 mg daily in 54 men with oligoasthenozoospermia. Men treated with SG1002 had increased sperm concentration, motility and motile forms recovery compared to those treated with placebo. Resveratrol had no effect on any of these parameters. During the study, three men in the SG1002 arm withdrew: one due to excessive flatulence and nausea, one due to “smelly” sweating, and one for convenience. No other adverse effects of SG1002 were reported.

The purpose of this trial was to evaluate initial safety and maximum tolerated oral doses of SG1002. This trial determined whether SG1002 is superior to placebo in increasing circulating hydrogen sulfide levels in healthy human subjects and those with CHF. The comparator in this study was placebo.

The primary objective of this trial was to determine the safety of SG1002. The secondary objective of this trial was:
To describe the safety of SG1002 in healthy subjects and subjects with CHF;
To describe the pharmacokinetics of SF1002 in healthy subjects and subjects with CHF; and
To determine whether oral doses of SG1002 can reverse hydrogen sulfide deficiency, assess changes in heart function as measured by brain natriuretic peptide (BNP) and reduce oxidative stress in healthy subjects and subjects with CHF.

This was a randomized, placebo-controlled double-blind dose escalation study. Six of eight of the healthy subjects were randomly assigned to SG1002. Once all eight healthy subjects completed the first dose level, ethics approval was sought to continue the study in patients with CHF. The next 8 CHF subjects were randomised in a 3:1 ratio (SG1002:placebo).

The Investigator did not carry out any deviations from, or changes to the protocol without agreement of the Sponsor and prior review and documented approval or favourable opinion from the IRB or IEC. The only exceptions were where it was necessary to remove an immediate hazard to the trial subjects, or when the changes were logistic or administrative (for example change in monitor or change of telephone numbers).

### Example 2: Study Design Methods

This study was conducted in Melbourne, Australia by a dedicated phase 1 unit (Nucleus Network). Healthy subjects met all the inclusion criteria, but none of the exclusion criteria, as listed below. Subjects met all of the following:

1. Healthy male volunteers aged between 18 and 45 years (inclusive);

2. Body mass index between 19 and 30 kg/m2 (inclusive);

3. No clinically significant findings in the medical history and physical examination;

4. No clinically significant laboratory values and urinalysis, unless the investigator considers any abnormality to be clinically irrelevant;

5. Normal ECG, blood pressure and heart rate, unless the Investigator considers any abnormality to be not clinically significant (NCS);

6. Willing to use contraception (single barrier methods); and

7. Willing and able to provide written informed consent.

The following criteria were not met:

1. Have received blood products within 1 month prior to Screening;

2. Have received any investigational research agent within 30 days or 5 half-lives (whichever is longer) prior to the first dose of trial medical food;

3. Have received an investigational vaccine within 6 months, a live attenuated vaccine within 60 days or a registered vaccine within 30 days prior to the first dose of the trial medical food;

4. Have a history of thyroidectomy or thyroid disease that required medication within the past 12 months;

5. Have had serious angioedema episodes within the previous 3 years or requiring medication in the previous two years;

6. Have a bleeding disorder diagnosed by a doctor (for example factor deficiency, coagulopathy, or platelet disorder requiring special precautions) or significant bruising or bleeding difficulties during blood draws;

7. Have a psychiatric condition that precludes compliance with the protocol, past or present psychoses, past or present bipolar disorder, or a disorder requiring lithium, within five years prior to enrolment;

8. Has a history of suicide plan;

9. Any clinically significant abnormality at Screening determined by medical history, physical examination, blood chemistry, haematology, urinalysis and a 12-lead ECG, or positive urine screen for drugs of abuse;

10. Any other condition which in the view of the Investigator is likely to interfere with the study or put the subject at risk;

11. HIV, or hepatitis B or C positive;

12. Have a history of or current clinically significant gastrointestinal, hepatic, renal, cardiovascular, respiratory, endocrine, oncological, immunodeficiency, neurological, metabolic, haematological or autoimmune disorder;

13. Have a history of or current tuberculosis, epilepsy, diabetes or glaucoma;

14. Have clinical signs of active infection or a temperature more than 38.0° C. at the time of screening. Study entry may be deferred at the discretion of the Principal Investigator;

15. Have evidence of drug or alcohol abuse;

16. Hypersensitivity to sulfur or related compounds;

17. Be unable to provide repeated blood samples without undue trauma or distress; or

18. Anticipate surgery within the trial period.

Congestive heart failure subjects (CHF) met all of the inclusion criteria, but none of the exclusion criteria as listed below. CHF subjects met the following criteria:

1. Aged between 35 and 85 years (inclusive);

2. Has symptomatic heart failure, with New York Heart Association (NYHA) classification of II or III;

3. Ambulatory;

4. Left ventricular ejection fraction less than 40%;

5. Normal haemoglobin at screening;

6. Congestive heart failure has been stable for the previous 3 months (defined by no change in baseline therapy or symptoms of heart failure for the previous 3 months); and

7. Willing and able to provide written informed consent.

The following criteria were not met in CHF subjects:

1. Subject is pregnant or breastfeeding;

2. If female, the subject is either post-menopausal or surgically sterilised or willing to use an acceptable method of birth control (i.e., a hormonal contraceptive, intrauterine device, diaphragm with spermicide, condom with spermicide, or abstinence) from signing of the informed consent form though to the Final Visit/Early Termination Visit;

3. Myocardial infarction, unstable angina, stroke, cerebrovascular accident, percutaneous coronary intervention, open heart surgery or transient ischemic attack (TIA) within 3 months prior to Screening;

4. Current symptomatic hypotension (defined as systolic blood pressure (SBP) 90 mmHg or diastolic blood pressure (DBP) 40 mmHg);

5. Poorly controlled hypertension (defined as SBP 160 mmHg or DBP 100 mmHg) despite therapy

6. Subjects with NYHA grade IV heart failure;

7. Subjects awaiting percutaneous coronary intervention or open heart surgery;

8. Subjects with serious liver disease;

9. Subjects with liver function tests three times the upper limit of normal;

10. Have a history of haemolytic anemia;

11. Any change in cardiovascular drug therapy within three months prior to randomization

12. History of chronic obstructive pulmonary disease (diagnosed using GOLD criteria) or x-ray evidence of restrictive lung disease (defined as FEV1 to FVC ratio of >80%);

13. Poorly controlled diabetes (defined as HbA1c>10.0%);

14. Has undergone Cardiac Resynchronisation Therapy (CRT) in the last 6 months and no planned CRT;

15. Implantable cardioverter defibrillator (ICD) implant planned during the study;

16. Hypersensitivity to sulfur or related compounds;

17. Renal insufficiency defined as eGFR<30 mL/minute/1.73 m2 (Modification of Diet in Renal Disease Study MDRD);

18. Life expectancy less than 6 months;

19. Active malignancy requiring active anti-neoplastic therapy that will, in the opinion of the investigator, interfere with study treatment or participation. (Stable basal cell skin cancer and cancers being treated solely with hormonal therapy are allowed);

20. Have evidence of drug or alcohol abuse;

21. HIV, or hepatitis B or C positive; or

22. Any other chronic illness that may, in the opinion of the Investigator, increase the risks associated with this trial.

Randomize subjects received either SG1002 or placebo as the trial medical food. This was a dose escalation study. Initially, patient were given 200 mg oral capsules SG1002 b.d. (twice daily) or matched placebo for 7 days (Visit 1); then increased to 400 mg SG1002 (2×200 mg oral capsules) b.d. or matched placebo for 7 days (Visit 2); then increased to 800 mg SG1002 (4×200 mg oral capsules) b.d. or matched placebo for 7 days (Visit 3). Doses were given with or without food.

Trial medical food was labelled according to local regulatory requirements. The trial medical food was packaged in child-proof bottles containing 16 capsules. Product blinding was preserved by means of suitable, yet traceable labelling. Do not store trial medical food above 30° C. Store in a secure area with restricted access. Once dispensed to subjects, store trial medical food was stored at or below 30° C.

The Sponsor provided the sites with enough trial medication for this study. A responsible person (for example a pharmacist) received deliveries of trial medical food from the Sponsor. All receipts were recorded in writing. Product was stored in a secure area under recommended storage condition. Trial medical food was used in accordance with the protocol. The Investigator ensured the integrity of the trial medical food prior to dispensing. The trial medical food was dispensed as provided by the Sponsor with no further repackaging.

The site pharmacist/Investigator(s) dispensed the trial medication only to subjects enrolled in this trial following the procedures set forth above. The site pharmacist/Investigator(s) was responsible for assuring the retrieval of all trial supplies from the subjects. Complete trial medical food accountability then seek approval. The unused trial medical food was returned to the sponsor or destroyed on site (if appropriate).

The Investigator(s) kept accurate and acceptable records including:
dates of receipt and return of medical food shipments
batch number
quantities received or returned
dates and amounts dispensed to or returned by trial subjects.
   Trial supplies were not destroyed without prior approval from the Sponsor. The Investigator/site pharmacist provided the monitor with access to the trial medical food and records for periodic review. Copies of the accountability records were provided in the trial master file. Trial medical food was not used for purposes other than defined in this protocol. Trial medical food was discontinued in subjects who experienced a serioud adverse event which in the judgment of the Principal Investigator or Medical Monitor was related to the trial medical food. Treatment adherence was determined by returned trial medical food counts. A subject was considered to be compliant with trial medical food if they took at least 80% of prescribed doses. The subjects were counseled on the importance of adherence if they had less than 100% of their medication. Participants with less than 80% compliance were counseled, and their participation in the trial was re-evaluated. Any possible discontinuation due to trial medical food non-adherence was discussed with the Sponsor. Subjects were given any treatment (except those explicitly excluded) which was considered necessary for the subject's welfare. Concomitant medications were reported in the Case Report Form (CRF). Generic names, start and stop dates, total daily dose, route and indication were recorded. Trade names were acceptable for combination medications.

All necessary and appropriate concomitant medications were allowed in this trial. A concomitant medication is any medication (other than trial medical food), including herbal and over-the-counter preparations, the subject took during the trial period. Concomitant medications in the CRF (case report form) were recorded (hydrogen sulfide may induce cytochrome P450, therefore it may decrease the half-life of medications metabolized by these enzymes. Careful monitoring of concomitant medications metabolized by these enzymes is recommended). There were no prohibited medications or care. However, alcohol was not permitted 24 hours prior to visits.

The study assessed the safety of SG1002. Safety assessments are highlighted in the Time and events schedule below. These include concomitant medication use, clinical laboratory tests, physical examination, vital signs, ECG, and adverse events.

TABLE 2
Time and Events Schedule

| | | Visit Name | | | | | |
|---|---|---|---|---|---|---|---|
| | | | | | | Visit 4 or Early | Follow-up |
| | | Screening | Visit 1 | Visit 2 | Visit 3 | Termination (ET) | Phone call |
| | | Timing (days) | | | | | |
| | | | | | | | Day 28 or at ET |
| | | Day −21 to −1 | Day 0 | Day 7 | Day 14 | Day 21 | plus 7 days |
| | | Allowable Variance | | | | | |
| | | | | ±1 day | ±1 day | ±1 day | ±2 days |
| Screening | Informed consent | X | | | | | |
| | Inclusion/exclusion review | X | X | X | X | X | |
| | Demographics | X | | | | | |
| | Medical and medication history | X | X^{a} | X^{a} | X^{a} | X^{a} | |
| Efficacy | Physical examination | X | X | X | X | X | |
| | Vital signs^{b} | X | X | X | X | X | |
| | ECG^{c} | X | X | X | X | X | |
| | Oxidative stress biomarker sampling^{d} | | X | X | X | X | |
| | Pharmacokinetic sample (time 0)^{c} | | | | | X | |
| | BNP sample^{c} | | X | X | X | X | |
| | Repeat pharmacokinetic sampling^{e} | | | X^{f} | X^{a} | | |
| Safety | Clinical safety laboratory samples^{h,i} | X | | X | X | X | |
| | Urine pregnancy test | X | X | X | X | X | |
| | Urine screen for drugs of abuse | X | | X | X | X | |
| | Adverse events | | X | X | X | X | X |
| | Evaluate study stopping criteria | | X | X | X | | |
| Trial medical | Dispense trial medical food | | X | X | X | | |
| food | Trial medical food accountability | | | X | X | X | |

^{a}Concomitant medication review only.
^{b}Vital signs should be assessed prior to SG1002 dose and at discharge for every visit.
^{c}Prior to SG1002 dose only.
^{d}Take a single sample at 0 hours.
^{e}Take samples at 0, 0.5, 1, 2, 4, 6, 12 and 24 hours after administration of trial medical food for healthy subjects.
^{f}Take samples at 0, 0.5, 1, 2 and 4 hours after administration of trial medical food for heart failure subjects.
^{g}Take samples at 0, 0,5, 1, 2, 4, 6, 12, and 24 hours after administration of trial medical food for heart failure subjects.
^{h}Blood and urine
ⁱHIV, Hepatitis B and C screening at baseline only

The laboratory assessments were performed at a local laboratory. The laboratory provided a list of laboratory reference ranges, units of measurement, and current laboratory certification to the Sponsor before beginning the trial. During the trial period, the laboratory must provide updates to the Sponsor, if required. The following laboratory assessments were obtained according to the Time and events schedule:
Clinical chemistry panel (sodium, potassium, chloride, bicarbonate, urea, creatinine, fasting glucose, calcium, total protein, albumin, alkaline phosphatase, alanine amino transferase, aspartate amino transferase and bilirubin);
Full blood count (haemoglobin, haematocrit, red cell count (RCC), mean corpuscular volume (MCV), mean corpuscular haemoglobin (MCH), mean corpuscular haemoglobin concentration (MCHC), red cell distribution width (RDW), platelets, white cell count (WCC), neutrophils, lymphocytes, monocytes, eosinophils, basophils);
Haemolytic blood tests: unconjugated bilirubin, LDH, haptoglobin and reticulocyte count. (If haemoglobin levels fall below normal levels, a blood film and Heinz body prep will be carried out);
Serology (HIV, Hepatitis B and C, performed at Screening only);
Urinalysis (haemoglobin, glucose, pH, protein, blood); and
Drug screen.

Clinically relevant abnormal clinical laboratory tests were reported as Adverse Events (see Example 4). Physical examinations were performed according to the Time and events schedule. A doctor conducted the physical examination, but where timing restricts this, a suitable trained nurse undertook this task. Doctors were consulted if any abnormality in the examination was detected. Clinically relevant changes in physical examination were reported as Adverse Events (see Example 4).

Oral temperature, pulse, respiratory rate, and blood pressure were assessed according to the Time and Events Schedule (once per visit). Seated blood pressure using an automatic device consisting of an inflatable cuff and oscillatory detection system was assessed. Clinically relevant changes were reported in vital signs as Adverse Events.

A twelve-lead surface electrocardiogram (ECG) was performed in the semi-supin position prior to SG-1002 administration on a recently serviced and calibrated ECG machine. Machine performance data was maintained in the trial master file.

The Investigator evaluated the effects of the trial medical food on the ECG. Clinically relevant changes in the ECG were reported as Adverse Events. Clinically relevant changes were tabulated and included:
trial medical food-induced prolongation of the QT/QTc interval;
Torsade de points;
ventricular tachycardia;
ventricular fibrillation and flutter;
syncope; or
seizures.
   Safety outcomes were collated and tabulated for the entire duration of the trial.

Secondary endpoints included plasma hydrogen sulfide levels, plasma nitrite levels, and oxidative stress biomarkers. The change from baseline to visit 4 for each were calculated. Plasma hydrogen sulfide levels were determined using the methods described in the laboratory manual. Plasma levels of BNP were assessed by the clinical laboratory to determine whether there were changes in heart function. Reduced glutathione (GSH) levels andoxidized glutathione (GSSG) levels were used as markers of oxidative stress. The ratio of GSH:GSSG were also determined. Methods of measuring GSH and GSSG are described in the laboratory manual. Plasma nitritelevels were determined using the methods described above.

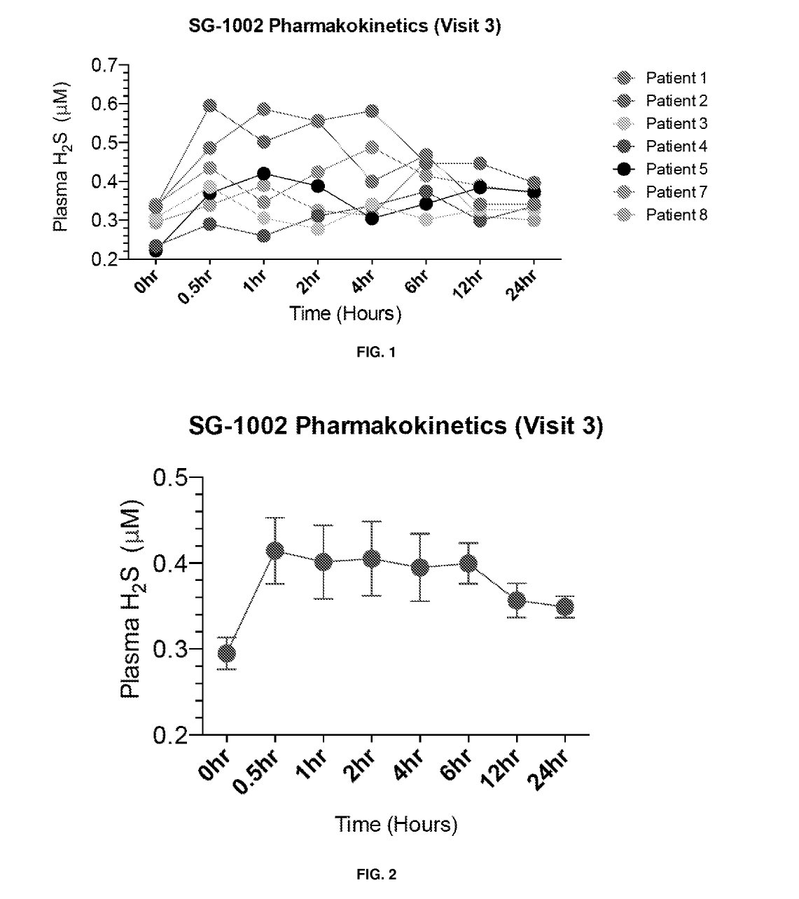

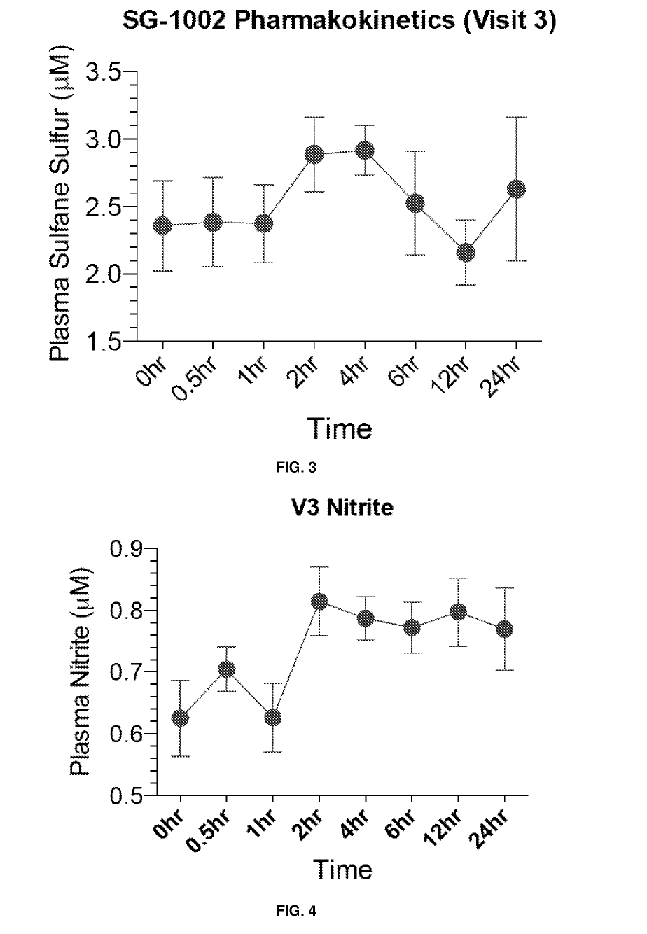

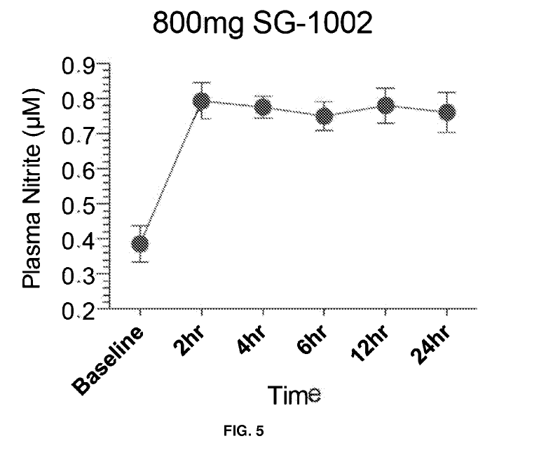

Pharmacokinetic samples were repeated to assess plasma hydrogen sulfide levels and plasma nitrite levels and taken at 0, 0.5, 1, 2, 4, 6, 12, and 24 hours after trial medical food on Days 0, 7, and 14 for healthy subjects (see, FIGS. 1-5). A single pharmacokinetic sample was taken on Day 21, or at early termination. Samples were tested to determine AUC, t_{1/2}, Cₘₐₓ and volume of distribution. For heart failure subjects, pharmacokinetics samples were repeated to assess plasma hydrogen sulfide levels and plasma nitrite levels at 0, 0.5, 1, 2, and 4 hours after tiral medical food on Days 0 and 7, and at 0, 0.5, 1, 2, 4, 6, 12, and 24 hours after trial medical food on Day 14. A single pharmacokinetic sample was taken on day 21, or at early termination. Samples were tested to determine AUC, t_{1/2}, Cₘₐₓ and volume of distribution.

Samples for analysis of BNP and oxidative stress were taken on Days 0, 7, 14, 21 and at early termination. Time 0 blood draws for pharmacokinetic, BNP and oxidative stress analysis should precede taking the trial medical food. Pharmacokinetic and oxidative stress biomarkers were compared between each visit. Comparisons to baseline was made at the beginning of each visit to determine whether there was any chronic sustained increase in hydrogen sulfide levels associated with trial medical food.

At least 16 subjects were included in this dose escalation study (at least eight healthy volunteers and eight patients with congestive heart failure). Healthy patients were randomized to SG1002 or placebo with a 3:1 ratio. Patients with congestive heart failure were randomized to SG1002 or placebo with a 3:1 ratio. Additional patients were recruited to account for drop-outs or non-compliance (an additional one healthy patient, and two patients with congestive heart failure).

Nucleus Network recruited the eight healthy volunteers from their database. If needed, potential subjects with congestive heart failure were identified from the Principal Investigators cardiology clinics, referrals from colleagues and advertising.

The project statistician and data management programmer produced the computer generated randomization code. The code was stratified such that the eight healthy participants were randomized 3:1 (medical food:placebo) and the subsequent 8 subjects with congestive heart failure are randomized 3:1 (medical food:placebo). Details of the method of producing the allocation sequence were defined in a separate document. Allocation was completed using the online Medrio Electronic Data Capture (EDC) system. The principal investigator (or designee) had blinded access to this system. A specially designated member of Nucleus Network was able to unblind a subject in the case of an emergency. George Clinical produced the allocation sequence. The Investigator enrolled subjects following informed consent and inclusion/exclusion assessment subjects. The Investigator assigned participants to interventions. Enrolled subjects were randomly assigned to one of two treatments using the Medrio EDC system. The pharmacist or Investigator dispensed the treatment allocated to the subject and recorded the kit number and enrollment number in the dispensing log. Treatment was not assigned to a subject before consent was given and all inclusion criteria and none of the exclusion criteria were met.

Both the subject and the investigative staff were blinded to the treatment allocation. The trial pharmacist or blinded Investigator and Nucleus Network kept the sealed randomization envelopes. The envelopes were only made available to the Principal Investigator when a serious adverse event required unblinding or in an emergency. Unblinding was also done using the eDC module by designated unblinded staff. The Sponsor was consulted before breaking any randomization code, unless it is an emergency.

### Example 3: Participant Timeline

The trial consisted of five trial visits and a follow-up telephone call over 28 days. Subjects were screened for eligibility during the Screening phase. Refer to the Time and events schedule for a list of procedures performed during the screening phase. Screening was performed between 1 to 21 days prior to Visit 1.
Obtained written informed consent. This was completed prior to any trial-related procedures.
Reviewed inclusion/exclusion criteria.
Obtained demographic information including: date of birth; height and weight (no shoes); sex; ethnicity.
Performed a physical exam.
Reviewed medical and medication history. Recorded concomitant medications for the previous 3 months.
Measure vital signs including oral temperature, pulse, respiratory rate and blood pressure.
Conducted ECG.
Collected blood sample and urine for clinical safety laboratory testing and drug screening. All blood sampling occurred after vital signs and ECG assessments.
Performed urine pregnancy test (for females of child-bearing potential only).

### Double-Blind Phase (Day 0-21)

During this phase the Investigator assessed subject response to therapy using the efficacy and safety measures outlined above. Refer to the Time and events schedule for a list of procedures performed during the double-blind phase. The double-blind phase involved visits on Day 0 (visit 1), Day 7 (Visit 2), Day 14 (Visit 3) and day 21 (Visit 4). A follow-up telephone call occurred on Day 28.

### Visit 1 (Day 0)

Refer to the Time and events schedule for a list of procedures performed during Visit 1.
Reviewed eligibility criteria;
Performed a physical exam;
Recorded concomitant medications that have changed since screening;
Measure vital signs including oral temperature, pulse, respiratory rate and blood pressure;
Conducted ECG;
Collected blood sample for BNP and oxidative stress testing. All blood sampling occurred after vital signs and ECG assessments;
Collected 5 mL blood samples at time 0, 0.5, 1, 2, 4, 6, 12 and 24 hours for pharmacokinetic analysis for healthy subjects. Collected 5 mL blood samples at time 0, 0.5, 1, 2, and 4 hours for pharmacokinetic analysis for heart failure subjects. Time 0 samples were taken immediately prior to administering the trial medical food.
Performed urine pregnancy test (for females of child-bearing potential only).
Collected and report adverse events.
Evaluated stopping criteria.
Dispensed trial medical food.

### Visit 2 (Day 7)

Refer to the Time and events schedule for a list of procedure performed during Visit 2.
Reviewed eligibility criteria;
Performed a physical exam;
Recorded concomitant medications that have changed since screening;
Measure vital signs including oral temperature, pulse, respiratory rate and blood pressure;
Conducted ECG;
Collected blood sample for BNP and oxidative stress testing. All blood sampling occurred after vital signs and ECG assessments;
Collected 5 mL blood samples at time 0, 0.5, 1, 2, 4, 6, 12 and 24 hours for pharmacokinetic analysis for healthy subjects. Collected 5 mL blood samples at time 0, 0.5, 1, 2, and 4 hours for pharmacokinetic analysis for heart failure subjects. Time 0 samples were taken immediately prior to administering the trial medical food.
Performed urine pregnancy test (for females of child-bearing potential only).
Collected and report adverse events.
Evaluated stopping criteria.
Dispensed trial medical food.

### Visit 3 (Day 14)

Refer to the Time and events schedule for a list of procedures performed during Visit 3. Complete the procedures outlined for Visit 2 with the

exception as follows:
Collected 5 mL blood samples at time 0, 0.5, 1, 2, 4, 6, 12 and 24 hours for pharmacokinetic analysis for all subjects. Time 0 samples were taken immediately prior to administering the trial medical food;

### Visit 4 (Day 21) or at Early Termination

Refer to the Time and events schedule for a list of procedures performed during Visit 4 and the early termination visit.
Reviewed eligibility criteria;
Performed a physical exam;
Recorded concomitant medications that have changed since the previous visit;
Measured vital signs including temperature, pulse, respiratory rate and blood pressure;
Conducted ECG;
Collected blood and urine sample for clinical safety laboratory testing, BNP and oxidative stress testing. All blood sampling occurred after vital signs and ECG assessments;
Collected 5 mL blood sample at time 0 for pharmacokinetic analysis. Time 0 samples were taken immediately prior to administering the trial medical food;
Performed urine pregnancy test (for females of child-bearing potential only);
Collected and report adverse events;
Evaluated stopping criteria;
Performed returned medication counts.

### Follow-Up Phone Call

Refer to the Time and events schedule for a list of procedures to be performed during the follow-up phone call.
Collected adverse events;
Reminded subjects to report any adverse events that occurred up to 30 days after the last dose of trial medical food.

Subjects were considered to have completed the trial if he or she completed assessments up to Visit 4 and completed the follow-up phone call phase or experienced a clinical endpoint that precluded further study. Subjects who prematurely stopped trial treatment for any reason before completion of the double-blind phase were not considered to have completed the trial.

Subjects were withdrawn from the trial if:
They became pregnant;
They experience a serious adverse event which in the opinion of the Principal Investigator or Medical Monitor was related to the trial medical food;
They were non-adherent with trial medical food;
They were lost to follow up;
They withdrew consent;
The Investigator believed that for safety reasons (for example adverse event, intercurrent illness) that it was in the best interests of the subject to withdraw;
The subject's doctor asked they withdraw; or
The Investigator or the Sponsor, for any reason, stopped the trial or stopped the subject's participation in the trial.

Protocol violations did not lead to automatic withdrawal unless they were a significant risk to the subject's safety. All protocol violations were promptly discussed with the Sponsor. A subject withdrew from the trial, in consultation with the Sponsor, if:
They did not adhere to trial medical food; or
They had poor visit attendance.
The Investigator completed and reported the observations up to the time of withdrawal.
   All subjects who were asked to withdraw underwent an Early Termination Visit. The reason and date of withdrawal from the trial were noted. The subject continued to be monitored if the reason for withdrawal was an adverse event (AE) or an abnormal laboratory test result until a proper medical judgment about the cause or importance was made. The event or test result was recorded on the CRF. Subjects who withdrew was replaced by an additional one healthy subject or up to two patients with congestive heart failure.

### Example 4: Adverse Events (AE) and Pregnancy

An AE was any untoward medical event in a subject given the trial medical food that does not necessarily have a causal relationship with the trial medical food. An AE was therefore any unfavourable or unintended sign (including an abnormal laboratory finding), symptom, or disease temporally associated with the use of the trial medical food, whether or not related to the trial medical food. Any worsening (for example clinically significant adverse change in frequency or intensity) of a pre-existing condition, which was temporally associated with the use of the trial medical food, was also an AE. Recurring symptoms were not considered associated with pre-existing conditions as AEs, unless they have a clinically significant increase in severity or frequency. Changes resulting from normal growth or development occurring at a physiologically appropriate time were not considered as AEs. AEs were identified by:
Information volunteered by the subjects (and/or caregivers or parents (if appropriate));
Open-ended and non-leading questions such as: Have you had any health problems since your last visit?; and
Observation by the investigational team, other care providers or relatives.
   The appropriate clinical staff at the trial site provided assessment and treatment of any trial related medical emergencies involving trial subjects.

A serious adverse event for this trial was defined as any untoward medical occurrence that at any dose: 1) results in death, 2) is life-threatening, 3) requires inpatient hospitalization or prolongation of existing hospitalization, 4) results in persistent or significant disability or incapacity, 5) is a congenital anomaly or birth defect; or 6) is an important medical event. Serious adverse events were identified by:
Information volunteered by the subjects (and/or caregivers or parents (if appropriate));
Open-ended and non-leading questions such as: Have you had any health problems since your last visit?; and
Observation by the investigational team, other care providers or relatives;
   The appropriate clinical staff at the trial site provided assessment and treatment of any trial related medical emergencies involving trial subjects.

The Investigator made a causality assessment according to his or her best judgment. The causal relationship was classified according to the following criteria listed in Table 3.

TABLE 3
AE causality assessment

| Label | Likelihood | Description |
|---|---|---|
| Definite | >95% | The evidence for the trial medical food causing |
| | | the event is beyond a reasonable doubt |
| Highly likely | 75-95% | The evidence for the trial medical food causing |
| | | the event is clear and convincing, but not |
| | | definite |
| Probable | 50-74% | Most evidence supports the link between the |
| | | trial medical food and the event |
| Possible | 25-49% | The evidence for the trial medical food causing |
| | | the event is equivocal but present |
| Unlikely | <25% | There is evidence that an aetiological factor |
| | | other than the trial medical food caused the |
| | | event |
| Unknown | — | The case is not informative enough to assign |
| | | the event to any of the above categories |

The Investigator made a severity assessment according to his or her best judgment. The severity was classified according to the criteria in Table 4.

TABLE 4
AE severity assessment

| Label | Definition |
|---|---|
| Mild | Awareness of sign or symptom but easily tolerated |
| | and does not alter normal activity |
| Moderate | Sign or symptom causes discomfort or interference |
| | with usual activity |
| Severe | Sign or symptom causes significant impairment of function |
| | or incapacitation, or inability to do usual activities |

All AEs were reported, whether serious or not, from signing the informed consent form until 30 days after the last dose of trial medical food. Only spontaneously reported adverse events that occurred between the follow-up phone call and 30 days after the last dose of trial medical food were reported. All Serious Adverse Events were reported, including those spontaneously reported to the Investigator by the subject within 30 days of the last dose of trial medication, using the Serious Adverse Event Form. Only spontaneously reported serious adverse events that occurred between the follow-up phone call and 30 days after the last dose of trial medical food were reported. All events that met the definition of a serious adverse event were reported as Serious Adverse Events. All AEs were recorded, regardless of seriousness, severity or presumed causality in the subject's medical records and in the CRF. The Investigator assessed all AEs and recorded details of seriousness, severity duration, action taken with trial medical food, and relationship to the trial medical food. The Investigator also recorded details of de-challenge and re-challenge with the trial medical food, if it were performed.

The Principal Investigator or designee reported all Serious Adverse Events (SAEs) within 24 hours after any member of the Investigational team becomes aware of the event. The Investigator assessed causality and severity of the Serious Adverse Events. All actions taken and outcomes were recorded.

Serious Adverse Events were followed up until:
the event resolved;
its sequalae resolved or stabilized at a level acceptable to the Investigator;
until the report was assessed as complete; or
the subject was lost to follow up.
   Specific extra follow up information was obtained for the Sponsor in an expedited fashion for full safety assessment. All follow up information was reported as described above. An SAE was immediately reported to the Sponsor any time after the 30-day post trial follow up period if it was considered to be related to the trial medication. Any event requiring hospitalisation (or prolongation of hospitalisation) that occurred during the trial period as a serious adverse event was reported, except hospitalisations for:
Social reasons
Surgery or procedure planned before entry into the trial (document in the CRF at trial entry).

Pregnancy was not considered an AE. The Sponsor was notified immediately if a subject, or the sexual partner of a male subject, becomes pregnant while they were taking part in the trial or within the 30-day post-trial follow up period. Trial medical food was stopped for female subjects who became pregnant. Follow up was continued for female subjects who became pregnant for scheduled safety assessments. All pregnancies were followed to term and the outcome reported to the Sponsor. Consent was given from the partner to get follow up information for a pregnancy in the sexual partner of a male subject.

### Example 5: Data Collection

Data was collected using an electronic CRF. The data was monitored by George Clinical on at least 4 occasions throughout the trial period. Nucleus Network ensured subject retention and complete follow-up (where possible) using their usual process, which included contacting the subject by telephone, email and registered post. Subjects who discontinued had outcome data collected as described above.

The monitor(s), auditor(s), and regulatory inspector(s) were given direct access to source data and documentation (for example medical records, laboratory reports, diagnostic imaging) for source data verification. Subject confidentiality was upheld following local requirements and privacy laws.

The Investigator(s) ensured all trial personnel were qualified for their appointed roles. He or she provided information about the trial to all staff members involved in the trial or in any element of subject management. Information was provided before starting the trial and during the trial (for example when new staff become involved). All persons appointed by the Investigator to participate in the trial was indicated on the delegation of authority log.

The monitor(s) initiated the site, ensured site compliance with the protocol and closed the site at the end of the trial. The monitor provided any new information available during the trial and helped train new staff members.

The protocol was read thoroughly and instructions were followed. Exceptions to the protocol were made in an emergency when the protection, safety, and well-being of the subject required immediate intervention. This decision was based on the judgment of the Investigator or a responsible, suitably trained, sub-investigator. The Investigator must telephone the Medical Monitor at the earliest possible if a significant protocol deviation due to an emergency, accident, or error occurred. This allowed for an early joint decision to be made as to whether the subject should continue in the trial. The Investigator, the Sponsor, and the Medical Monitor documented this decision.

### Example 6: Data Management

George Clinical generated a data management plan. The Investigator prepared and kept adequate and accurate medical records. These records contained all observations and other data relevant to the trial for each trial participant. Enough information must be contained to allow for verification of subject identity throughout the trial. The Sponsors defined any data in the CRF for which no other written or electronic record was maintained. This data was considered as source data (for example results of physical examinations, vital signs testing, or the medical food administration procedure). The monitor, auditors, representative from the IRB or IEC, and regulatory bodies reviewed the CRF and the subject's medical records related to the trial, if allowed by local laws. Regulatory bodies included the US Food and Drug Administration (FDA). The Investigator kept a subject identification code list to allow unambiguous identification of each subject included in the trial. This list contained the subject's full name, date of birth, dates of participation and identification number according to country regulations. A note was made in the hospital or clinical medical records, if appropriate, the subject was a part in a clinical trial. At the end of the trial the Investigator supplied all data to Sponsor auditor(s) or regulatory authorities.

In the trial the Case Report Form was electronic (Medrio database). The Investigator or designee completed the CRF and supporting documentation for each subject in a timely manner of the visit occurring. The monitor(s) and data manager(s) reviewed the completed data for accuracy, completeness and consistency with source documentation. The monitor or data manager requested correction or clarification of data when inconsistencies were identified. The Investigator or designee completed corrections and alterations of CRF data in a timely manner. The Principal Investigator (or designee) reviewed and signed completed CRFs. A full audit trail detailing corrections and alternations made to the CRF was maintained.

Trial data and other essential documents was kept for at least:
Fifteen years for studies in adults;
Twenty-five years for studies in children;
Two years after the last approval of a marketing application; or
Two years after the formal discontinuation of clinical development of the trial
medical food.
   The Sponsor told the Investigator(s)/institution(s) when these documents can be destroyed. The Sponsor archived the original CRFs lifetime of the trial medical food product. Any trial document was not to be destroyed without prior written agreement between the Sponsor and the Investigator(s). Should the Investigator(s) wish to assign the trial records to another party or move them to another location, advanced written notice was given to the Sponsor.

### Example 7: Statistical Methods and Monitoring

A separate Statistical Analysis Plan included a full description of the statistical methods planned for this trial. Demographic data, clinical chemistry, complete blood counts, biomarkers, and adverse events were summarized in tabular form by dose level and overall. Descriptive statistics was used to summarize the demographic and clinical data. Laboratory values above and below the normal limit were flagged, and adverse events presented by System Organ Class (SOC), severity and relationship to study treatment. Pharmacokinetics models were used to estimate concentration parameters for each dose.

Analysis of the primary endpoint was performed on a full population set basis and was repeated in the per protocol (PP) population. Results from the full population analysis took precedence over findings from the PP analysis. Other measures were performed using the full population, and where applicable, the PP population. No adjusted analyses was performed. The full analysis set was included for all subjects who enrolled. The per protocol analysis set was included for all subjects who enrolled and completed all follow-up visits and were adherent to the trial medical food. Missing data was not imputed. Patients who withdrew from the study were replaced. No interim analysis was planned.

A data monitoring committee was set up comprising of the members from Sulfagenix's Clinical Advisory Board. The data monitoring committees duties were:
Reviewing significant AEs and AE trends;
Examining outcome and safety data to recommend continuing, stopping or changing the trial;
Reviewing major trial design changes; and
Reviewing the general progress of the study.
   The data monitoring committee had access to SAE and periodic safety reports in real-time blinded to treatment allocation.

The expected monitoring frequency was four visits over the six-month recruitment and treatment period, but was subject to recruitment rates. The monitor(s) ensured the trial was conducted according to Standard Operating Procedures (SOPs) and ensured compliance with GCP. The monitor(s) was the primary link between Sponsor and Investigator. The monitor(s) visited the Investigator to ensure adherence to the protocol; to assure that all data were correctly recorded; and to check informed consent was obtained for all subjects before their participation in the trial. The monitor(s) contacted and visited the Investigator regularly throughout the trial. The monitor(s) were allowed to check and verify the various records (CRFs and other relevant data records) relating to the trial. This allowed them to verify adherence to the protocol and to ensure the completeness, consistency, and accuracy of the data recorded. As part of supervising the trial progress other Sponsor personnel may, on request, go with the monitor(s) on visits to the trial centre. The Investigator and designee agreed to cooperate with the monitor(s) to resolve any data discrepancies detected during these monitoring visits.

The Sponsor may audit the investigational site to compare raw data, source data, and associated records with the interim (if applicable) or final report of the trial to assure that data have been accurately reported. Auditing may be contracted to an external body. The Investigator(s) accepted that regulatory authorities may conduct an inspection to verify compliance of the trial with GCP.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents and patent applications are herein incorporated by reference in their entirety to the same extent as if each individual publication, patent or patent application was specifically and individually indicated to be incorporated by reference in its entirety.

1. A method of treating a nitric oxide (NO) deficiency disorder in a nitric oxide-deficient subject, said method comprising providing a sulfur composition in an amount and for a duration sufficient to treat said NO deficiency disorder in said subject, wherein said NO deficiency disorder is selected from the group consisting of: endothelial dysfunction, argininosuccinic aciduria, Huntington's disease, sickle cell disease, hyperhomocystinemia, acute chest syndrome, muscular dystrophy, dyslipidemia, pre-eclampsia, and Alzheimer's disease.

2. A method of supplementing deficits in circulating nitrite in a nitric oxide deficient subject, said method comprising administering a sulfur composition in an amount and for a duration sufficient to treat said deficit in circulating nitrite in said subject.

3. A method of improving bodily endurance or exercise performance in a subject in need thereof, said method comprising administering an effective amount of a sulfur composition, wherein said composition is administered to said subject prior to or during exercise.

4. The method of claim 3, wherein said composition is administered to said subject within 0-180 minutes prior to exercise.

5. The method of claim 3, wherein said composition is administered to said subject during exercise.

6. The method of claim 1, wherein the level of circulating nitrite in said subject is less than about 0.3 μM in the fasted state.

7.-10. (canceled)

11. The method of claim 6, wherein the level of circulating nitrite is between about 0.1 μM to about 0.3 μM in the fasted state.

12. The method of claim 1, wherein administration of said composition results in an increase in plasma nitrite concentration within about two hours after administration of said composition.

13. The method of claim 1, wherein said plasma nitrite concentration is maintained between about 0.5 μM to about 1 μM for 2-12 hours after administration of said composition.

14. The method of claim 1, wherein administration of said composition to said subject provides for gradual release of said sulfur composition over 4-14 hours.

15. The method of claim 1, wherein said sulfur composition comprises at least one of hydrogen sulfide, sodium hydrogen sulfide, sodium sulfide, potassium sulfide, calcium sulfide, sodium hydrosulfide dihydrate, sodium sulfide nonahydrate, ammonium sulfide, sodium thiosulfate, potassium thiosulfate, sodium thiosulfate pentahydrate, magnesium thiosulfate, silver thiosulfate, ammonium thiosulfate, calcium dithionate, barium dithionate dihydrate, sodium trithionate, sodium tetrathionate, zinc sulfoxylate, zinc dithionite, sodium dithionite, sodium dithionite dehydrate, or elemental alpha sulfur and highly polar components.

16. The method of claim 1, wherein said composition is formulated for enteral, topical, or parenteral administration.

17. The method of claim 16, wherein said composition is formulated for enteral administration and said sulfur composition is present in an amount of about 200 mg to about 800 mg.

18. The method of claim 17, wherein said composition is a capsule.

19. The method of claim 16, wherein said composition is formulated for topical administration and said composition comprises from about 1% to about 20% sulfur composition.

20. (canceled)

21. The method of claim 1, wherein said composition is administered between one to four times a day.

22. The method of claim 1, wherein said composition is administered for at least two to ten days.

23. The method of claim 1, further comprising administering a second agent.

24. The method of claim 23, wherein said second agent is a cardiovascular disease drug, an anti-inflammatory drug, an anti-neurodegenerative drug, or an anti-cancer/anti-proliferative drug.

25. The method of claim 23, wherein said second agent is a dietary supplement selected from the group consisting of: a vitamin, a micronutrient, coenzyme Q10, glucosamine, chondroitin sulfate, vinpocetine, pramiracetam, diallyl sulfide, N-acetylcysteine, pyridoxal-s-phosphate, methylcobolamin, 5-methylcetrahydrofolate, nitrate, a concentrate, an amino acid, an herb or botanical, and a mineral.

26.-28. (canceled)
